(19) United States
(12) Patent Application Publication
(10) Pub. No.: US 2010/0004443 A1
(43) Pub. Date: Jan. 7, 2010

(54) LINEAR PYRIDAZINE AND PYRROLE COMPOUNDS, METHOD FOR OBTAINING THEM AND APPLICATIONS

(75) Inventors: Didier Max Dubreuil, Port Saint Pere (FR); Muriel Geneviève Pipelier, Nentes (FR); Jean-Paul Pradere, Sautron (FR); Hicham Bakkali, Nantes (FR); Christine Geneviève Juliette Thobie, Treillieres (FR); Eric Fredéric Leonel, Chatillon (FR); Jean-Yves Joseph Maria Nedelec, L'hay-les-roses (FR); Stephane Sengmany, Vitry Sur Seine (FR); Thierry Delaunay, Sable-sur-Sarthe (FR); Alexandra Tabatchnik, La Baule (FR)

Correspondence Address: NIXON & VANDERHYE, PC, 901 NORTH GLEBE ROAD, 11TH FLOOR, ARLINGTON, VA, 22203, US

(73) Assignee: CENTRE NATIONAL DE LA RECHERCHE SCIENTIFIQUE (C.N., Paris Cedex 16 (FR)

(21) Appl. No.: 12/309,632

(22) PCT Filed: Jul. 26, 2007
(86) PCT No.: PCT/FR2007/001288

§ 371 Date: May 21, 2009

(30) Foreign Application Priority Data
Jul. 26, 2006 (FR) 06/06841

(51) Int. Cl. C07D 401/14

(52) U.S. Cl. 544/238

(57) ABSTRACT

The present invention relates to linear pyridazine compounds, and more particularly to those of these compounds which are oligopyridazine compounds, to processes for obtaining them, to their uses, and also to their reduction to pyrroles and to the uses of the pyrrole, pyridazinylpyrrole and oligopyrrole compounds obtained. The invention relates in particular to the uses as medicaments, in particular for treating pathologies such as cancer, bacterial infections or parasitic infections, and also the applications in the materials, environmental, electronics and optics field.

This invention relates to linear pyridazine compounds, more particularly to oligopyridazine compounds, to methods for obtaining them and applications thereof, as well as to their regression into pyrroles, and to the various applications of pyrrole, pyridazine-pyrrole and oligopyrrole compounds. The terms “oligopyridazine” and “oligopyrrole” denotes compounds with a plurality of adjacent nitrogen-containing rings.

Since the end of the 80's, coordination chemistry applying to nitrogenous compounds has known considerable developments; these are due to the diversity of chemical and catalytic properties exhibited by organometallic compounds having one or more nitrogen functions in their coordination spheres. This diversity is mainly associated with the nitrogen functions involved in these complexes: amine, imine, nitrile, azide, etc.

Coordination chemistry plays a fundamental role in supramolecular chemistry, a domain in which oligopyridines first attracted special attention. Oligopyridines are polydentate ligands that can also be classified according to the number of nitrogen atoms involved in metal chelation in this complex: bidentates (bipyridines), tridentates (terpyridines), tetradentates (quaterpyridines), etc., with the following structures:

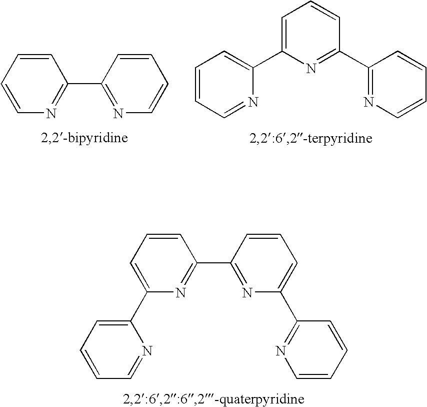

2,2′-bipyridines have long been the ligands most frequently used in coordination chemistry, especially when they exhibit the asymmetric induction properties associated with the presence of chirality factor-inducing groups. More recently, 2,2′:6′,2″-terpyridines (tpy) have opened a new field of investigation with the expression of polydentate sites that are favourable to the formation of complexes with transition metals of higher oxidation states. This property has been used, for instance, for the oxidation of alcohols and the carbonylation of aromatic compounds. Even more recently, the chemistry of this type of polydentate ligands has been further developed in terms of catalytic activation for the remediation of radioactive waste.

Apart from the heterogeneous structures consisting of pyridine and pyrimidine units that express a variety of polydentate coordination sites, relatively little work has been done on oligoheterocyclic ligands including diazine units.

Examples of such ligand structures are shown hereunder:

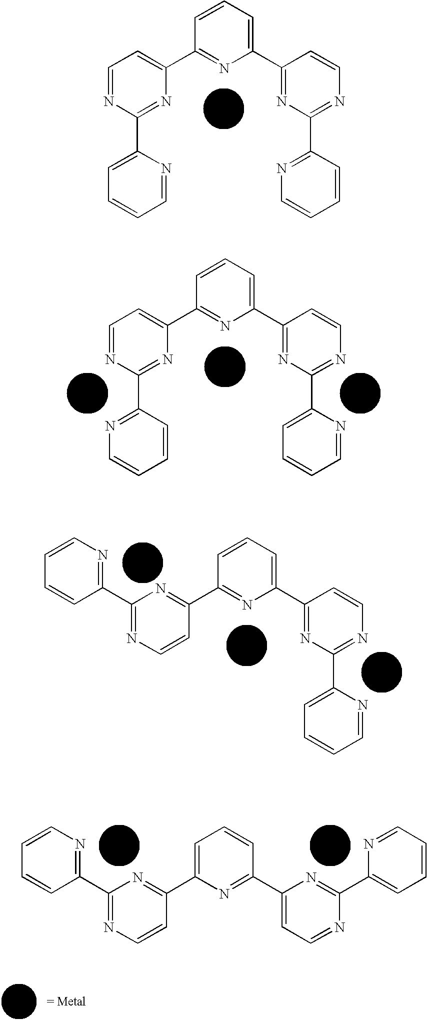

As in the mentioned work done on the 3,6-bis(pyridin-2-yl)-pyridazines 80 bidentate ligands (Hoogenboom, R. et al., Eur. J. Org. Chem., 2003, p. 4887; see scheme hereunder), polydentate ligands with pyridazine heterocyclic rings have only been developed very recently, because of the delicate synthetic process they require, although their potential in coordination chemistry is now obvious.

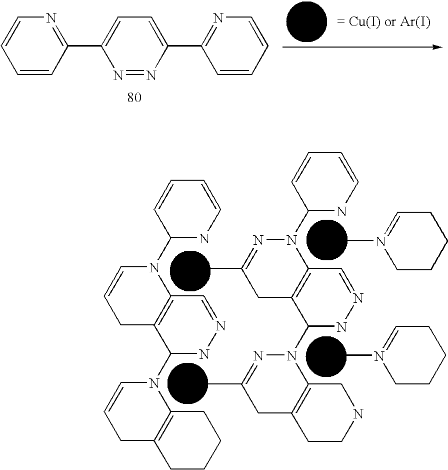

Furthermore, 6,6′-bis(6-methylpyridin-2-yl)-3,3′-bipyridazine 2 has demonstrated strong supramolecular organisation capacities in the presence of various metals, such as silver (I) (Baxter, P. N. W.; Lehn, J.-M., Fisher, J.; Youinou, M.-T. Angew. Chem., 1994, 106, 2432).

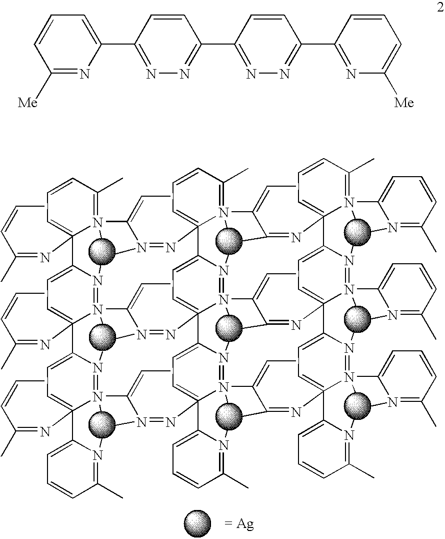

In 2000, the same authors managed to synthesise a pyridazine tetramer from a dichlorobipyridazine 17 precursor, through homocoupling of halogenated bipyridazines (Baxter, P. N. W; Lehn, J.-M.; Baum, G.; Fenske, D. Chem. Eur. J., 2000, 6, 4510).

Just like its dimeric homologue, this α,α′-tetrapyridazine ligand has a linear geometry and can therefore only express sequences of poly-bidentate coordination sites for one molecule. In the presence of silver, this tetramer (quaterpyridazine) will preferentially provide a supramolecular arrangement of a square-grid type, as is the case with bipyridazine 2. However, the elongation of the pyridazine chain also results, in this case, in the self-arrangement of four monomers that results in a helix organisation made of a tetramer balanced with the square grid.

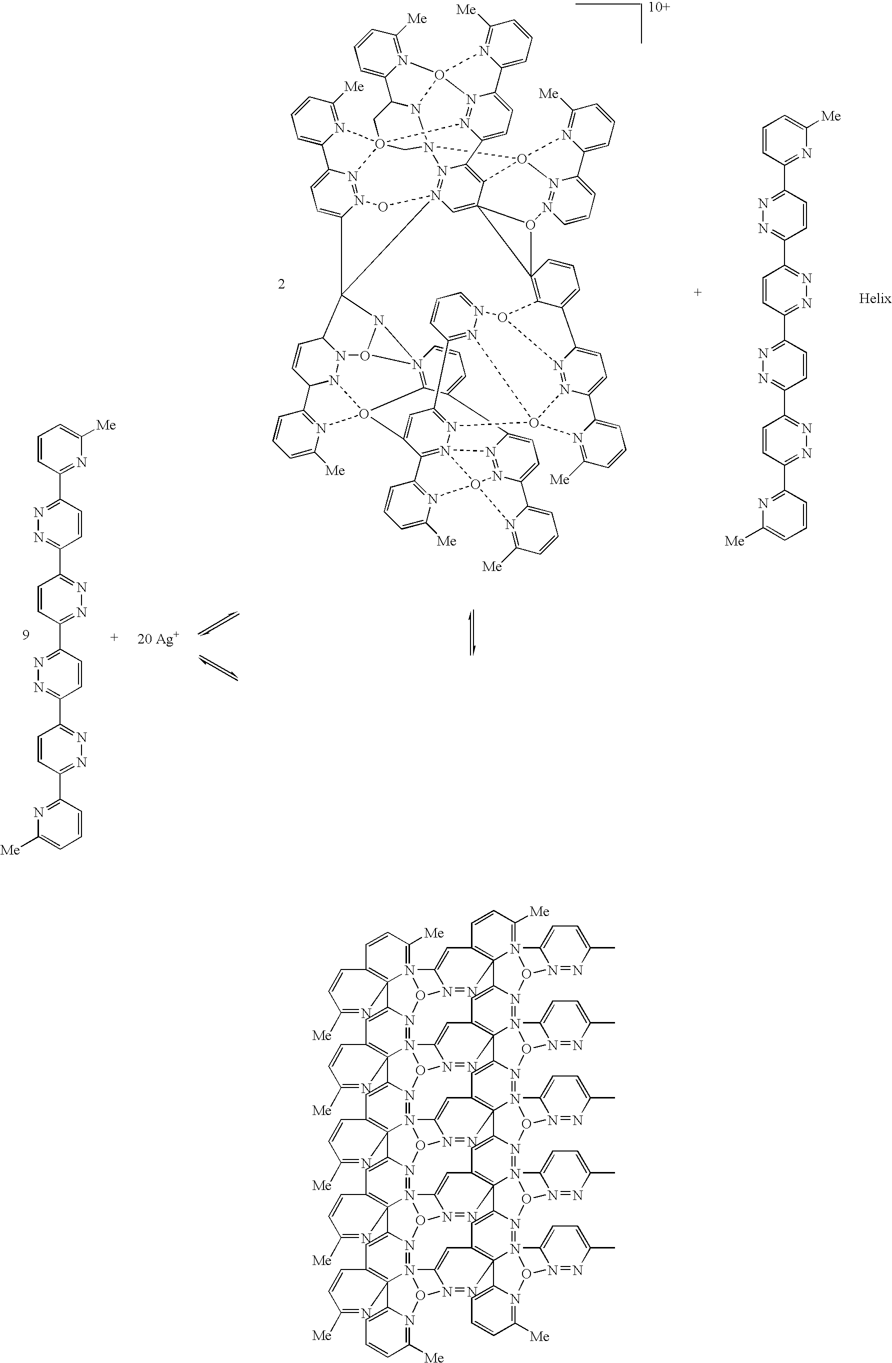

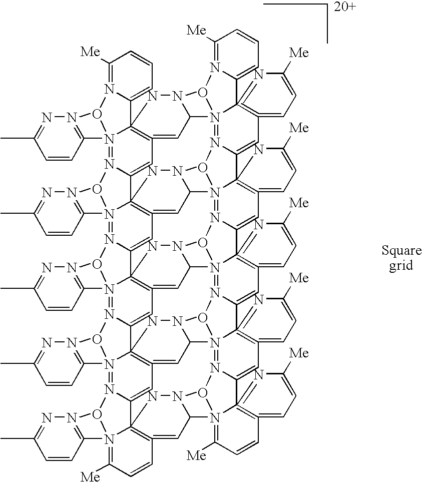

Given the high potential of the compounds described above, and given the lack of a standardisable route for the synthesis of these compounds and their analogues—which are not numerous—, the inventors have decided to pave the way for a new generation of pyridazine and pyrrole ligands.

Yet, in previous work, the inventors devised a method for electrochemically reducing monopyridazines into monopyrroles (Manh G. T. et al., Electrochimica Acta, 2002, 2833).

The inventors have therefore hypothesised that an electrochemical reduction of oligopyridazine compounds can be achieved under conditions that are to be determined, despite the presence of a plurality of adjacent pyridazine rings that are likely to strongly modify the structural and electronic properties of the molecule.

In the first step for the validation of this hypothesis, the inventors had to devise routes for synthesising oligopyridazine compounds. The synthetic routes that were developed are numerous. One of them relates in particular to an optimisation of the synthetic route proposed by Lehn et al., in the aforementioned publication. This synthesis is purely organic, but others have been achieved electrochemically.

Furthermore, thanks to this chemical synthetic route, new substituted bispyridinyl-pyridazine compounds, and in particular asymmetric compounds, have been prepared.

Once the oligopyridazine compounds were ready, an oligopyrrole reduction could be tempted. Unexpectedly, this reduction is not only effective under the specific conditions devised and optimised by the inventors, but it does not give rise to cyclisation of the pyridazine residues or any other potential secondary reaction. Furthermore, the reduction can be also achieved pyridazine ring after pyridazine ring, thus providing one or more reduction site(s) on the molecule, and paving the way for the preparation of mixed pyridazinyl-pyrrole compounds.

The inventors also endeavoured to identify the potential biological applications of these new compounds. These compounds turned out to have very interesting therapeutic properties, in particular anti-parasitic, anti-cancer, and anti-bacterial properties.

This invention therefore relates to linear pyridazine sequences, more particularly to substituted bispyridinyl-pyridazine and oligopyridazine compounds, to methods for obtaining them and to applications thereof, as well as to the electrochemical oligopyridazine-to-oligopyrrole reduction process; and to the pyrroles, more particularly the bispyridinyl-pyrroles and oligopyrroles obtained, and to applications thereof.

In a first aspect, the invention relates to compounds having the formula

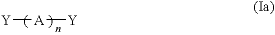

in which
if n=1
   if A is a group of the formula

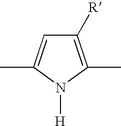

in which R′ is hydrogen, an alkyl, hydroxyalkyl, alkylamine, alkyloxy chain with 1-6 carbon atoms, a —COOH, —COOR1, —CONH₂, —CONHR1 group in which R1 is an alkyl chain with 1-6 carbon atoms,
the Y groups, which are identical or different, represent a group of the formula

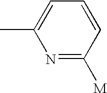

in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if A is a group of the formula

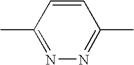

the Y groups, which are identical, represent a group of the formula

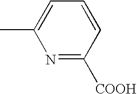

or the Y groups, which are different, represent a group of the formula

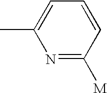

in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if n is an integer from 2 to 4, both inclusive,
   the A groups, which are identical or different, represent a group of the formula

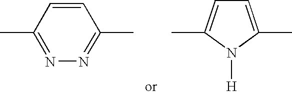

the Y groups, which are identical or different, represent halogen, hydroxy, mercapto, an alkyl, hydroxyalkyl, alkylamine or alkyloxy chain with 1-6 carbon atoms, optionally cyclic, a —COOH, —COOR1, —CONH₂, —CONHR1 group in which R1 is as defined above, or a group selected from:

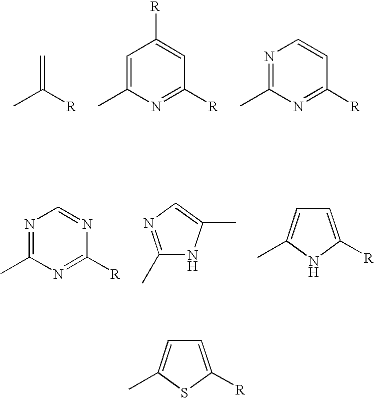

in which the R groups, which are identical or different, represent hydrogen, an alkyl or alkyloxy chain with 1-6 carbon atoms, a —COOH, —COOR1, —CONH₂, —CONHR1 group in which R1 is as defined above,
   with the exception of the following compounds:
2,5-bis(pyridin-2-yl)pyrrole,
6,6′-bis(6-methylpyridin-2-yl)-3,3′-bipyridazine,
6,6′″-bis-(6-methylpyridin-2-yl)-[
3,3′:6′,6″:3″,3′″]quaterpyridazine,
6,6′-dimethoxy-3,3′-bipyridazine,
6,6′-dichloro-3,3′-bipyridazine.

The preparation of the bispyridinyl-mono- and oligopyridazine compounds is described hereunder. Mono- and oligopyrrole compounds and the corresponding pyridazinyl-pyrrole compounds are obtained by means of an optimisation of the reduction process described in Manh G. T. et al., Electrochimica Acta, 2002, 2833.

The R′-substituted monopyrrole compounds are also prepared by electrochemical reduction, from R′-substituted pyridazine compounds, which is already described in the background art. In this case, the pyridazine compounds are obtained using the standard Diels-Alder reaction between a bipyridyl-tetrazine and an acetylene (see e.g. Hoogenboom et al., aforementioned).

However, some monopyridazines as well as the oligopyridazines and the corresponding oligopyrroles are entirely prepared using the methods of the invention, which are described hereunder.

The alkyl, hydroxyalkyl, alkylamine and alkyloxy chains are preferably methyl, hydroxymethyl, methylamine and methoxy groups, and the A groups are identical.

The invention relates more particularly to compounds in which, when n=1, M is hydrogen, halogen, an alkyl chain with 1-6 carbon atoms and a —COOH group, and preferably to the following compounds:
3-(2-carboxypyridin-6-yl)-6-(pyridin-2-yl)-pyridazine,
3,6-bis(2-carboxypyridin-6-yl)-pyridazine,
3-(6-methylpyridin-2-yl)-6-(pyridin-2-yl)-pyridazine,
3-(2-bromopyridin-6-yl)-6-(pyridin-2-yl)-pyridazine.

These compounds are described in further detail in the following Examples, in particular Examples 6-7 and 9-11.

When n=2, the Y groups, which are identical or different, represent preferably 2-pyridinyl groups, optionally substituted, or C(CH₂)OR1 groups in which R1 is an alkyl chain with 1-6 carbons, preferably an ethyl chain.

The following compounds are specifically described in Examples 1-5 and 8:
5,5′-bis(6-methylpyridin-2-yl)-2,2′-bipyrrole,
6,6′-di-(1-ethoxyvinyl)-3,3′-bipyridazine,
6,6′-bis(pyridin-2-yl)-3,3′-bipyridazine,
5,5′-bis(pyridin-2-yl)-2,2′-bipyrrole,
3-[5-(6-methylpyridin-2-yl)-pyrrol-2-yl]-6-(6-methylpyridin-2-yl)-pyridazine,
6-(pyridin-2-yl)-3-[(5-pyridin-2-yl)-pyrrol-2-yl]-pyridazine,
6,6′-bis(4,6-dimethylpyridin-2-yl)-3,3′-bipyridazine,
5,5′-bis(4,6-dimethylpyridin-2-yl)-2,2′-bipyrrole,
6-(4,6-dimethylpyridin-2-yl)-3-{[5-(4,6-dimethylpyridin-2-yl)-pyrrol-2-yl]-pyridazine}.

Herein, the terms “oligopyridazines” and “oligopyridazine compounds” on one hand, and “oligopyrroles” and “oligopyrrole compounds” on the other hand are used interchangeably. These terms denote compounds with a plurality of adjacent rings, preferably 2-4 rings.

In a second aspect, the invention relates to methods for obtaining the aforementioned compounds.

The first method within the scope of the invention is a method for preparing compounds having the formula

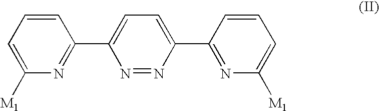

in which the M₁ substituents, which are identical or different, represent hydrogen, halogen, an alkyl or alkyloxy chain with 1-6 carbons,
by Stille coupling of a compound of the formula

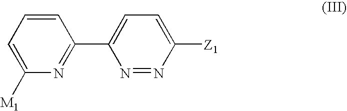

with a compound of the formula

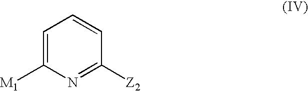

in which, Z₁ and Z₂, which are different, represent either a halogen atom or a stannylated group having the formula SnB₃, in which B is a methyl, butyl or phenyl chain.

The Stille-coupling reaction is a palladium (0)-catalysed coupling reaction that generates a carbon-carbon bond. The palladium (0) is preferably introduced in the reaction medium in the form of tetrakistriphenylphosphine palladium. The reaction is carried out under reflux in an organic solvent, preferably toluene, DMF (dimethylformamide), THF (tetrahydrofuran), HMPA (hexamethylphosphorotriamide), N-methylpyrrolidine.

The advantage of this synthetic route is that asymmetric pyridazine compounds are obtained (bipyridin-2-yl) by selecting the substituents on the pyridine rings.

The introduction of carboxylic groups from the compounds according to the invention is then performed by means of the following process, which provides a mono- or dicarboxylated compound. This is therefore a method for preparing compounds having the formula

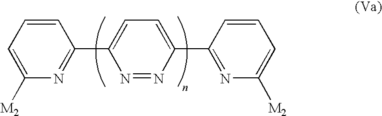

in which n is an integer from 1 to 4, both inclusive, at least one of the M₂ substituents is a —COOH group, the other substituent can be alternatively hydrogen, halogen or an alkyloxy chain with 1-6 carbon atoms, by oxidation of the methyl precursor in the presence of an allylic or aromatic oxidant such as selenium dioxide or chromium oxide.

Advantageously, the solvent used is a standard solvent for oxidation reactions, such as o-dichlorobenzene. The preferred temperatures at which the reaction is carried out range from 100 to 160° C., preferentially from 120 to 140° C.

The invention also provides a method for preparing compounds having the formula

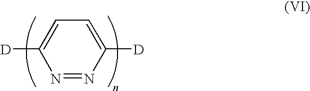

in which the D groups are halogen or an alkyloxy chain with 1-6 carbon atoms, preferably a methoxy or ethoxy chain, and n is an integer from 2 to 4, both inclusive, by coupling of at 1-east two halopyridazines having the formula

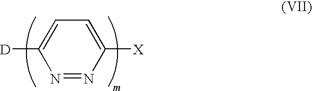

in which X is halogen, D is as defined above, and m is an integer from 1 to 3, both inclusive,
in the presence of a stoichiometric mixture of zinc, nickel dibromobis(triphenylphosphine) and tetrabutylammonium iodide in distilled and degassed dimethylformamide;
in which the coupling reaction is followed by a stage of purification by decomplexation.

The halogen functions are the reactive functions in this coupling reaction.

The reactions occur between 50 and 60° C.

This method is an alternative to the method proposed by Lehn et al., for the synthesis of 6,6′-bis(6-methylpyridin-2-yl)-3,3′-bipyridazine.

The optimised conditions are, in particular, the preparation in advance of the nickel dibromobis(triphenylphosphine), instead of its generation in situ in the reaction medium, as well as the use of tetrabutylammonium iodide. Advantageously, the nickel dibromobis(triphenylphosphine) and the tetrabutylammonium iodide are introduced at a ratio 1:0.3:1. In the Examples hereunder, the reaction is carried out at a temperature between 50 and 70° C.

Advantageously, in this method, the number of pyridazine rings in the oligopyridazine molecule can be incremented.

The purification stage is necessary for the decomplexation of the reaction product in the medium. The purification process can be achieved by either one of two distinct procedures.

In a first procedure, the purification of the compounds is carried out by decomplexation of said compounds in a cold aqueous solution saturated with potassium or sodium cyanide for 1.5-4 hours, preferably for 2-3 hours.

By “cold” we mean temperatures ranging from 0 to 25° C., preferably from 18 to 20° C.

In a second procedure, compound purification is achieved by decomplexation of said compounds in an aqueous solution saturated with potassium halide or tetrabutylammonium halide, but preferably potassium fluoride, or in saturated ammonia in which the organic phase is then washed with sodium or potassium hydrogencarbonate, and extracted with chloroform, dichloromethane, ethyl acetate, ether, etc.

The invention also relates to a method for preparing compounds having the formula

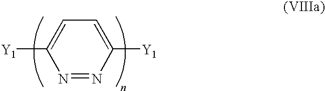

in which
if n=1,
the Y₁ groups, which are different, represent a group of the formula

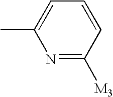

in which M₃ is hydrogen, an alkyl or alkyloxy chain with 1-6 carbon atoms,
if n is an integer from 2 to 4, both inclusive,
the Y₁ groups, which are identical or different, represent an alkyl or alkyloxy chain with 1-6 carbon atoms, or a group selected from:

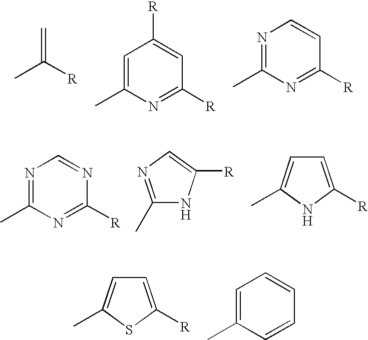

in which the R groups, which are identical or different, represent hydrogen, an alkyl or alkyloxy chain with 1-6 carbon atoms,
   by Stille coupling, at a ratio that ranges from 1:2 to 1:3, of a compound having the formula

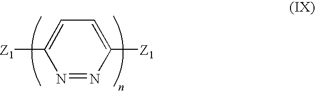

with a compound having the formula Y₁-Z₂
in which, Z₁, Z₂, which are different, represent either a halogen atom or a stannylated group having the formula SnB₃, in which B is a methyl, butyl or phenyl chain.

The invention also provides two preparation methods inspired from the Negishi coupling process.

This is the method used for preparing a compound of the formula

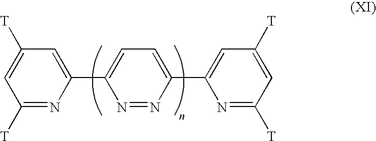

in which n is an integer from 1 to 4, both inclusive,
in which the T groups, which are identical or different, represent hydrogen, an alkyl chain with 1-6 carbon atoms, by coupling of a compound of the formula

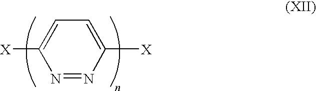

in which the X groups are halogen, and n is as defined above,
with a compound of the formula

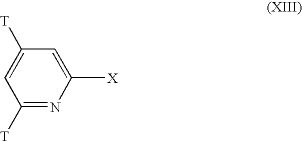

in which X and T are as defined above,
in the presence of butyllithium, a solvent, a zinc-based reagent and palladium (0).

According to this method, double coupling is possible, and the inventors have also devised a selective coupling method for preparing a compound of the formula

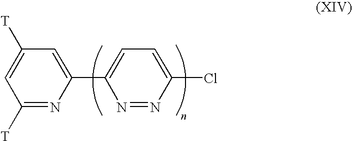

in which n is an integer from 1 to 4, both inclusive,
in which the T groups, which are identical or different, represent hydrogen, an alkyl chain with 1-6 carbon atoms, by selective coupling of a compound of the formula

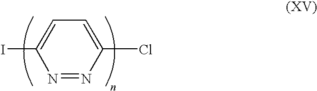

in which n is as defined above,
with a compound having the formula

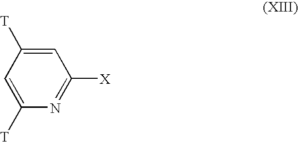

in which X is halogen,
in the presence of butyllithium, a solvent, a zinc-based reagent and palladium (0).

Advantageously, in these two methods, the solvent is THF or ether, the zinc-based reagent is ZnCl₂, and the palladium (0) is (Pd(Ph₃)₄) or Pd₂ dba₃.

The invention also provides the preparation of a new precursor: 3-methoxy-6-(pyridin-2-yl)-pyridazine.

This molecule is involved in two new methods: a method for preparing 3-methoxy-6-(pyridin-2-yl)-pyridazine by coupling of 3-chloro-6-methoxypyridazine with 2-trialkylstannylpyridine in the presence of palladium (0), and a method for preparing 6-(pyridin-2-yl)-2H-pyridazin-3-one by hydrolysis of 3-methoxy-6-(pyridin-2-yl)-pyridazine.

The invention also relates to a method for preparing 6,6′-bis(pyridin-2-yl)-3,3′-bipyridazine through the homocoupling of 3-chloro-6-(pyridin-2-yl)-pyridazine in the presence of dibromobistriphenylphosphine.

As an alternative to these synthetic routes, which are purely organic in nature, the inventors have also devised electrochemical synthetic routes.

The invention therefore provides a method for the electrochemical homocoupling of a pyridazine halide having the formula

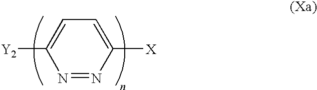

in which n is an integer from 1 to 2, both inclusive, X is halogen, and Y₂ is halogen, an alkyl or alkyloxy chain with 1-6 carbon atoms, a group selected from:

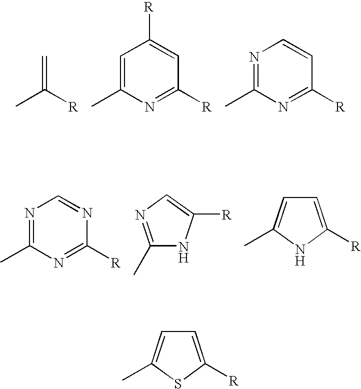

in which the R groups, which are identical or different, represent hydrogen, an alkyl or alkyloxy chain with 1-6 carbon atoms, or phenyl,
under the following hydrolysis conditions:
the anode is made of at least 50% iron,
the electrolysis medium contains nickel, an element selected from halogens, and pyridine or one of its derivatives.

Preferably, the anode used is a Fe/Ni (64/36) anode. Advantageously, the solvent of the reaction contains at least 50% DMF and a polar co-solvent. For instance, a mixture of dimethylformamide (DMF) and pyridine may be used, at a ratio ranging from 90/10 to 50/50, both inclusive, preferably of 80/20. The catalyst that is preferentially used is a nickel complex, such as a hydrated nickel halide. When the solvent does not contain any pyridine, advantage may be gained by using a nickel bipyridine halide as catalyst.

The support electrolyte is preferably a tetrabutylammonium halide or an equivalent such as tetrabutylammonium tetrafluoroborate, in a molar concentration ranging from 10 to 20%, both inclusive, preferably from 13 to 17%, in relation to the pyridazine substrate.

The amperage used for the reaction is e.g. from 0.05 A to 0.2 A, both inclusive, and preferably from 0.06 A to 0.1 A. The reaction can be conducted at room temperature (generally 20-25° C.).

Furthermore, the invention also provides a method for the electrochemical heterocoupling of a pyridazine halide having the formula

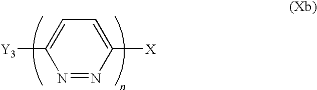

in which n is an integer from 1 to 2, both inclusive, X is halogen, and Y₃ is halogen, an alkyl or alkyloxy chain with 1-6 carbon atoms, or a group selected from:

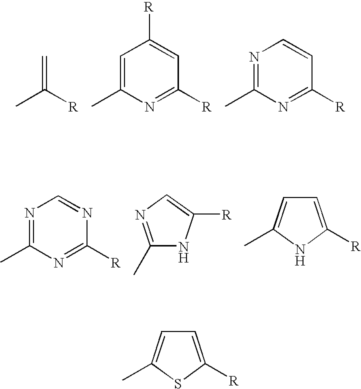

in which the R groups, which are identical or different, represent hydrogen, an alkyl or alkyloxy chain with 1-6 carbon atoms, or phenyl,
with a halide including an aromatic ring of the formula Ar—X, in which X is as defined above, and Ar is an aromatic ring with 5-6 links, optionally substituted, under the following electrolysis conditions:
the anode is made of iron,
the catalyst is selected from nickel bipyridine halides.

This reaction is specifically described in Example 3 hereunder.

Preferably, the solvent used is DMF while the support electrolyte is preferably a tetrabutylammonium halide or an equivalent such as tetrabutylammonium tetrafluoroborate, in a molar concentration from 10 to 20%, both inclusive, preferably from 13 to 17%, in relation to the pyridazine substrate. The amperage used for the reaction is e.g. from 0.15 A to 0.35 A, both inclusive, and preferably around 0.2 A. The reaction can be conducted at room temperature (generally 20-25° C.).

The aromatic ring is preferably a phenyl, pyridine or thiophenyl ring, optionally substituted.

The oligopyridazine-to-oligopyrrole reduction process that is the basis of the inventors' work is described hereunder. Examples 4 and 5 relate specifically to this method.

This method provides the pyrrole reduction of a compound having the formula

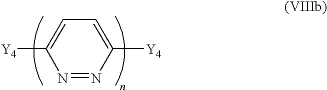

in which n is an integer from 2 to 4, both inclusive, the Y₄ groups, which are identical or different, represent an alkyl or alkyloxy chain with 1-6 carbon atoms, or a group selected from:

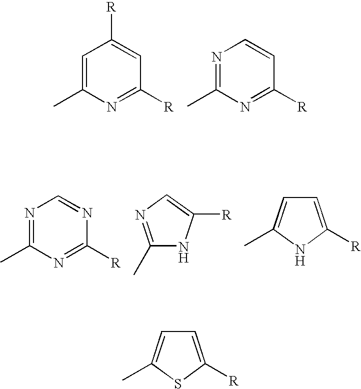

in which the R groups, which are identical or different, represent hydrogen, an alkyl or alkyloxy chain with 1-6 carbon atoms, or phenyl,
electrochemically, by extrusion of a nitrogen atom onto one or more pyridazine ring(s),
under the following electrolysis conditions:
the anode is an electrode with a large area,
the electrolysis medium is a proton-donating polar medium.

By way of example, the proton-donating polar medium may be made of an organic polar solvent (such as DMF, acetonitrile, etc.) completed with a proton donor (such a phenol, acetic acid, etc.), and optionally, when the resulting medium is not conductive, a support electrolyte such as quaternary ammonium salts or an acid-alcohol aqueous medium.

Advantageously, the quaternary ammonium salts are selected from tetrabutylammonium hexafluorophosphate, or tetrabutylammonium hydrogensulfate, and the acid-alcohol medium is a mixture of sulphuric or acetic acid and ethanol.

These media are the object of detailed description in Examples 4-8.

Preferably, the cathode is selected from mercury-film electrodes measuring 4.5 cm in diameter, large-area carbon electrodes, or screen-printed carbon electrodes.

The amperage that is used ranges between 10 and 50 mA. The reaction is conducted at room temperature (generally 20-25° C.).

The imposed reduction potential that varies with the considered substrates has to be controlled so as to control the consumption of Coulombs during the electrolysis, i.e. the number of electrons used, which is 4 for monopyrrole, 8 for bipyrrole, etc.

This method completes the work carried out by the inventors on the reduction of a pyridazine ring into a pyrrole (Manh G. T. et al., Electrochimica Acta, 2002, 2833).

Unexpectedly, this electrochemical reduction—that works on monopyridazines—turned out to be effective also on oligopyridazine compounds, under appropriate reaction conditions.

It was not easy to achieve a regression of the pyridazine rings because of the modification of the electronic environment of the rings; this modification is mainly due to the fact that the pyridazine rings that had to react in the molecule were by then adjacent to other identical rings that were also likely to react. Furthermore, this modification was also likely to generate synthesis intermediates with different electroreduction properties. Moreover, in the case where a regression could be achieved, it became likely that the electrochemical reduction steps, carried out simultaneously on several adjacent pyridazine structures, would interact and lead e.g. to possible degradations or internal rearrangements, or to partial reductions giving di- or tetrahydropyridazine intermediates instead of the pyrrole sequences. In the case of the regression turning out to be sequential (pyridazine ring after pyridazine ring), the aim was to estimate the influence of the possible formation of a first pyrrole or of a dihydropyridazine intermediate on the reduction potential of the mixed systems present at that moment.

By “regression” we mean two steps of reduction in an acid medium, the regression being the mechanistic result of electrochemical reductions.

Thanks to their work, the inventors have devised a procedure for the reduction of oligopyridazine compounds, and have demonstrated that this reduction is either simultaneous or sequential in nature, depending on the number of electrons and the potential applied during the electroreduction process.

With the regression process, new minority products have also been synthesised. These products are the following:
6-(4,6-dimethylpyridin-2-yl)-3-[5-(4,6-dimethylpyridin-2-yl)-1H-pyrrol-2-yl]-1,4,5,6-tetrahydropyridazine,
6-(6-methylpyridin-2-yl)-3-[5-(6-methylpyridin-2-yl)-1H-pyrrol-2-yl]-1,4,5,6-tetrahydropyridazine,
6-(pyridin-2-yl)-3-[5-(pyridin-2-yl)-1H-pyrrol-2-yl]-1,4,5,6-tetrahydropyridazine.

In all aforementioned methods, reference is made to alkyl and alkyloxy chains with 1-6 carbon atoms. Advantageously, said chains contain 1-3 carbon atoms, and are preferably methyl, ethyl, methoxy or ethoxy chains.

According to a third aspect, an object of the invention is to encompass the multiple applications of the synthesised compounds.

The compounds according to the invention are particularly well adapted to be used as ligands.

In particular, these compounds are ligands that complex particularly well with metal ions, in particular as far as iron, copper, ruthenium, europium, silver and bismuth cations are concerned. They may be used alone or as several identical ligands associated with each other.

As an example of non-limiting ligands, metal catenanes generated from compounds according to the invention are to be mentioned.

Furthermore, the inventors have researched the possible biological properties of the compounds, and identified interesting therapeutic properties.

The invention therefore relates to compounds having the formula

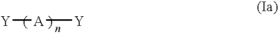

in which
if n=1
   if A is a group of the formula

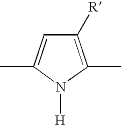

in which R′ is hydrogen, an alkyl, hydroxyalkyl, alkylamine, alkyloxy chain with 1-6 carbon atoms, a —COOH, —COOR1, —CONH₂, —CONHR1 group in which R1 is an alkyl chain with 1-6 carbon atoms,
the Y groups, which are identical or different, represent a group of the formula

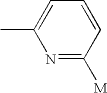

in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if A is a group of the formula

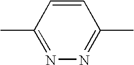

the Y groups, which are identical, represent a group of the formula

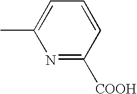

or the Y groups, which are different, represent a group of the formula

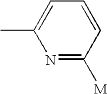

in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if n is an integer from 2 to 4, both inclusive,
   the A groups, which are identical or different, represent a group of the formula

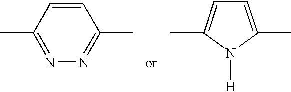

the Y groups, which are identical or different, represent halogen, hydroxy, mercapto, an alkyl, hydroxyalkyl, alkylamine or alkyloxy chain with 1-6 carbon atoms, optionally cyclic, a —COOH, —COOR1, —CONH₂, —CONHR1 group in which R1 is as defined above, or a group selected from:

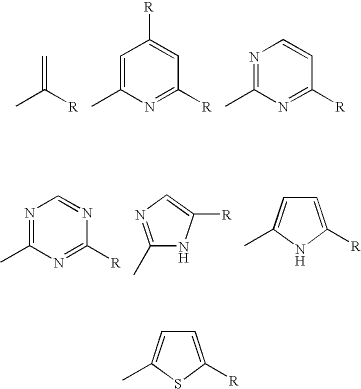

in which the R groups, which are identical or different, represent hydrogen, an alkyl or hydroxyalkyl chain with 1-6 carbon atoms, or phenyl, a —COOH, —COOR1, —CONH₂, —CONHR1 group in which R1 is as defined above,
   for use as drugs.

Preferably, the alkyl, hydroxyalkyl, alkylamine or alkyloxy chains with 1-6 carbons are methyl, hydroxymethyl, methylamine or methoxy chains.

Herein, the compounds listed above and the preferred compounds given hereunder are referred to as “compounds usable as drugs”.

More preferably, in the above formula, n is 2 and Y is a 2-pyridinyl group, optionally substituted, or a —C(CH₂)OR1 group in which R1 is an alkyl chain with 1-6 carbon atoms.

In fact, most of the compounds usable as drugs are new, and those that are not do not seem to have been studied in terms of their therapeutic properties.

The invention specifically encompasses the following compounds:
5,5′-bis(6-methylpyridin-2-yl)-2,2′-bipyrrole,
6,6′-di-(1-ethoxyvinyl)-3,3′-bipyridazine,
6,6′-bis(pyridin-2-yl)-3,3′-bipyridazine,
5,5′-bis(pyridin-2-yl)-2,2′-bipyrrole,
3-[5-(6-methylpyridin-2-yl)-pyrrol-2-yl]-6-(6-methylpyridin-2-yl)-pyridazine,
6,6′-bis(6-methylpyridin-2-yl)-3,3′-bipyridazine,
6,6′″-bis-(6-methylpyridin-2-yl)-[3,3′:6′,6″:3″,3′″]quaterpyridazine,
6-(pyridin-2-yl)-3-[(5-pyridin-2-yl)-pyrrol-2-yl]-pyridazine,
6,6′-bis(4,6-dimethylpyridin-2-yl)-3,3′-bipyridazine,
5,5′-bis(4,6-dimethylpyridin-2-yl)-2,2′-bipyrrole,
6-(4,6-dimethylpyridin-2-yl)-3-{[5-(4,6-dimethylpyridin-2-yl)-pyrrol-2-yl]-pyridazine}
   for use as drugs.

The therapeutic interest of these compounds is described in further detail in Example 42 hereunder. The invention also provides therapeutic compositions having, as active ingredients, the present compounds usable as drugs.

The invention also provides the present compounds usable as drugs in which n=1 and the M groups are halogen, an alkyl chain with 1-6 carbon atoms, or a —COOH group, for use as drugs.

Advantageously, the M groups, which are identical or different, represent halogen, an alkyl chain with 1-6 carbon atoms, or a —COOH group.

The invention specifically encompasses the following compounds:
3-(2-carboxypyridin-6-yl)-6-(pyridin-2-yl)-pyridazine,
3,6-bis(2-carboxypyridin-6-yl)-pyridazine,
3-(6-methylpyridin-2-yl)-6-(pyridin-2-yl)-pyridazine,
3-(2-bromopyridin-6-yl)-6-(pyridin-2-yl)-pyridazine,
2,5-bis(pyridin-2-yl)pyrrole,
   for use as drugs.

More particularly, the present compounds usable as drugs have several biological applications that can result in therapeutic applications.

In a first application, the present compounds usable as drugs can selectively complex with nucleic acids. In particular, they may be used as selective complexation agents of DNAs and RNAs, among which HIV. They act on the reverse transcriptase of the cells by inhibiting its primer on viral RNA. They are therefore particularly well suited for the preparation of anti-viral drugs. These compounds may also be used as DNA restriction agents (metallonuclease), particularly when they are complexed with a Cu-type metal.

In a second application, the present compounds usable as drugs have a cytotoxic activity towards cancerous cells. They are therefore particularly well suited for the preparation of anti-cancer drugs.

Preferably, the cancer concerned by the invention is a carcinoma, for instance a carcinoma of the ear, nose or throat, of the lungs, of the uterus, of the digestive system (oesophagus, colon, liver), of the skin, of the breasts, of the prostate, of the ovaries, etc. Specifically, 6,6′-di-(1-ethoxyvinyl)-3,3′-bipyridazine and 6-(pyridin-2-yl)-2H-pyridazin-3-thione, 3,6-bispyridin-2-ylpyridazine and 3-(6-methylpyridin-2-yl)-6-pyridin-2-ylpyridazine have shown very high levels of cytotoxic activity in a cancerous cell model: an in vitro test on KB, Caco, Huh7 and fibroblast cells.

In a third application, the present compounds usable as drugs act on the regulation of iron transfer in bacteria by inhibiting the tonB protein.

Iron is indispensable to bacterial infection. Yet, bacteria must retrieve the iron they need from their environment. Iron is always coupled with proteins such as transferrin, lactoferrin, haemoglobin, etc. Consequently, bacteria have very elaborate systems that involve the TonB protein to retrieve the iron.

In particular, 6,6′-di-(1-ethoxyvinyl)-3,3′-bipyridazine turned out to be able to inhibit the growth of E. coli.

These compounds are therefore particularly well suited for the preparation of antibacterial drugs, for example for treating dysentery or meningitis.

In a fourth application, the present compounds usable as drugs are also used to develop drugs for the treatment of parasitic diseases. The parasitic diseases concerned by the invention are in particular leishmanioses, aspergilloses and candidoses.

For this specific therapeutic application, advantageous use is made of compounds having the formula

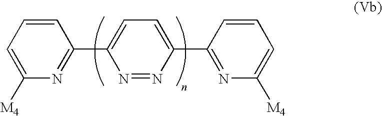

in which
n is an integer from 1 to 4, both inclusive,
the M₄ groups, which are identical or different, represent hydrogen, an alkyl chain with 1-6 carbon atoms, a —COOH, —COOR1 group in which R1 is an alkyl chain with 1-6 carbon atoms,
   and more particularly the compounds listed below:
3-(6-methylpyridin-2-yl)-6-(pyridin-2-yl)-pyridazine,
3-(2-carboxypyridin-6-yl)-6-(pyridin-2-yl)-pyridazine,
6,6′-bis(pyridin-2-yl)-3,3′-bipyridazine,
6,6′-bis(6-methylpyridin-2-yl)-3,3′-bipyridazine, and
3,6-bis(2-carboxypyridin-6-yl)-pyridazine.

In a fifth application, the present compounds usable as drugs are vectors of radioactive metals of great interest in terms of radioligands. Consequently, when they are complexed with the appropriate metal, such as bismuth or europium, they provide a drug for radioimmunotherapy.

For this specific therapeutic application, the preferred compounds are represented by formulae in which the A groups are groups of the formula

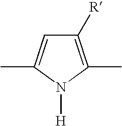

in which R′ is hydrogen, an alkyl, hydroxyalkyl, alkylamine or alkyloxy chain with 1-6 carbon atoms, a —COOH, —COOR1, —CONH₂, —CONHR1 group in which R1 is an alkyl chain with 1-6 carbon atoms.

These compounds are tridentate or tetradentate, N,O-mixed or N-donating ligands. They are therefore particularly well suited for the complexation of metal ions.

The inventors have also defined applications in the fields of environment, materials and electronics.

The compounds according to the invention may be advantageously used for the remediation of cations in liquid media. This applies specifically to compounds represented by formulae in which

A is a group of the formula

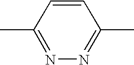

in which n is from 2 to 4, both inclusive, and
the Y groups, which are identical or different, represents hydroxy, a hydroxyalkyl or alkyloxy chain with 1-6 carbon atoms, optionally cyclic, a —COOH, —COOR1 group in which R1 is an alkyl chain with 1-6 carbon atoms, —CONH₂.

As mentioned hereinabove, these compounds are particularly well suited for the complexation of metal ions. They may possibly be used alone or as several identical ligands associated with each other.

Better still, the compound is selected from:
5,5′-bis(6-methylpyridin-2-yl)-2,2′-bipyrrole,
6,6′-di-(1-ethoxyvinyl)-3,3′-bipyridazine,
6,6′-bis(pyridin-2-yl)-3,3′-bipyridazine,
5,5′-bis(pyridin-2-yl)-2,2′-bipyrrole,
3-[5-(6-methylpyridin-2-yl)-pyrrol-2-yl]-6-(6-methylpyridin-2-yl)-pyridazine,
3-(2-carboxypyridin-6-yl)-6-(pyridin-2-yl)-pyridazine,
3,6-bis(2-carboxypyridin-6-yl)-pyridazine,
3-(6-methylpyridin-2-yl)-6-(pyridin-2-yl)-pyridazine, and
3-(2-bromopyridin-6-yl)-6-(pyridin-2-yl)-pyridazine.

Advantage may be gained from us of the compound according to the invention in combination with a carboxylic acid, in particular α-bromocapric acid. The inventors noted the presence of synergy in the remediation activities whenever this specific combination was implemented in relation, specifically, to actinide cations.

The invention also encompasses materials having a supramolecular organisation of compounds according to the invention. In particular, some compounds according to the invention exhibit self-assembly properties. Others can self-assemble around metal cations.

Furthermore, these materials also exhibit advantageous linear optics properties. In particular, they make it possible to develop liquid crystals, optical fibres, etc.

Other characteristics and advantages of the invention will be made clear in the following Examples, with reference to the drawings, in which, respectively:

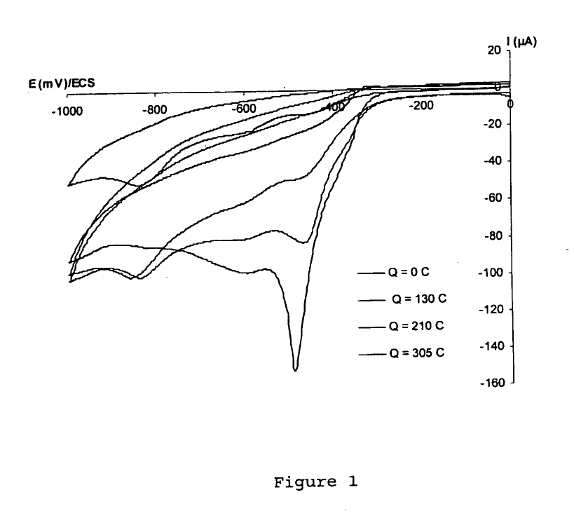

FIG. 1 shows cyclic voltamperogrammes during the preparative electrolysis, medium: 0.5 mol.L⁻¹H₂SO₄, ethanol (1/1), C=6.10⁻³ mol/L, V=100 mV/s;

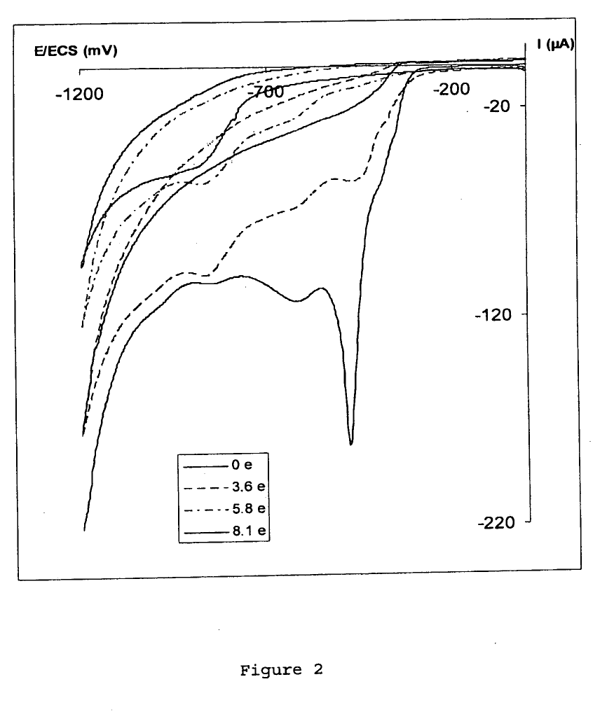

FIG. 2 shows cyclic voltamperogrammes during the preparative electrolysis of 6,6′-bis(6-methylpyridin-2-yl)-3,3′-bipyridazine 2 (—before electrolysis, —during electrolysis, —at the end of the electrolysis), vitreous carbon electrode; V=100 mV/s;

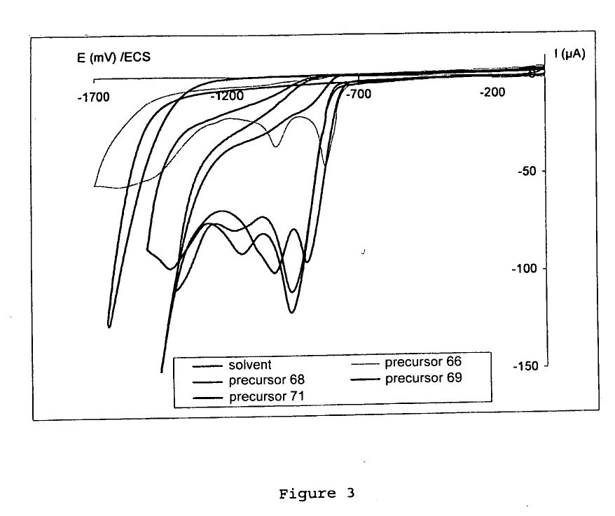

FIG. 3 shows cyclic voltamperogrammes of the solvent and of 3,6-bis(pyridin-2-yl)-pyridazines in an acetic acid/ethanol buffer medium, C=10⁻³ mol.L⁻¹, V=100 mV/s;

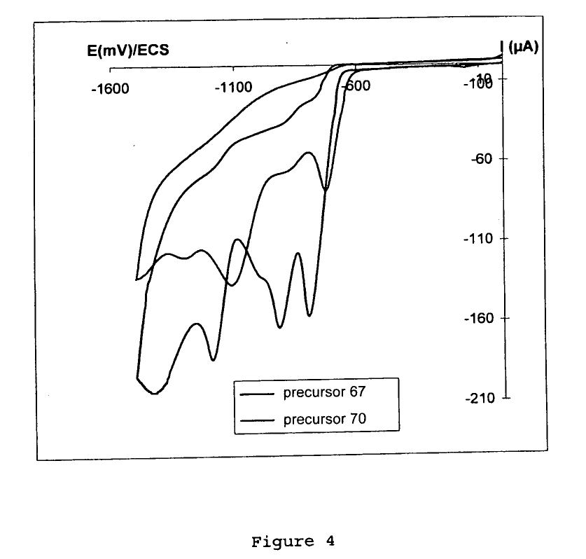

FIG. 4 shows cyclic voltamperogrammes of 4-carbomethoxy-3,6-bis(pyridin-2-yl)-pyridazine 84 and of 3,6-bis(pyridin-4-yl)-pyridazine 81 in an acetic acid/ethanol buffer medium, C=10⁻³ mol.L⁻¹, v=100 mV/s;

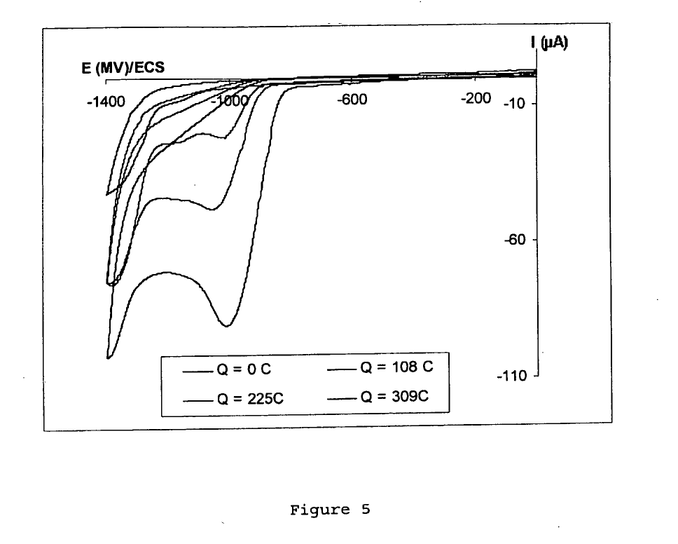

FIG. 5 shows cyclic voltamperogrammes during the preparative electrolysis of 4-(1-hydroxyethyl)-3,6-bis(pyridin-2-yl)-pyridazine 82 (—before electrolysis, —during electrolysis, —at the end of the electrolysis), vitreous carbon electrode, V=100 mV/s;

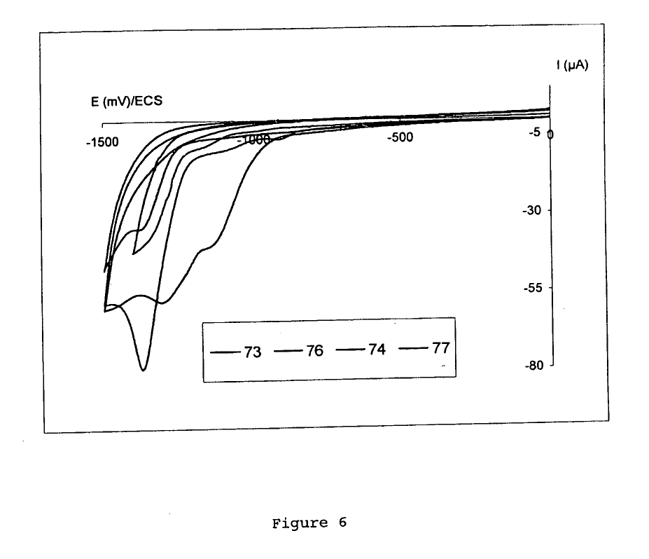

FIG. 6 shows voltamperogrammes of the various pyrrole derivatives at the end of the preparative electrolysis in the cathode compartment, vitreous carbon electrode, V=100 mV/s;

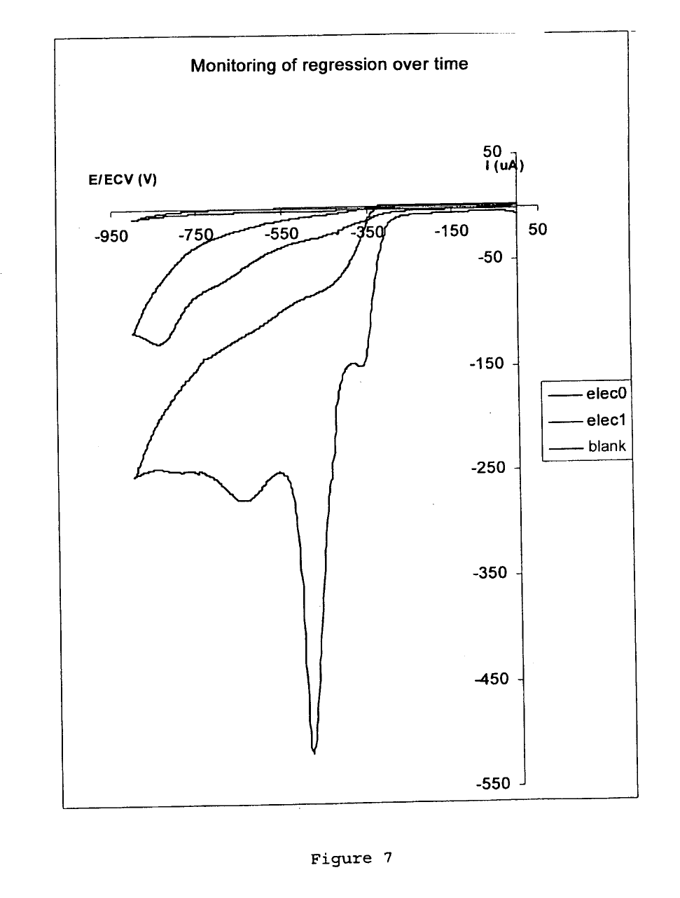

FIG. 7 shows a voltamperogramme of the preparative electrolysis of 6,6′-bis(pyridin-2-yl)-3,3′-bipyridazine (19), blank=reference voltamperogramme in the absence of the product, elec 0=control voltamperogramme at time 0, elec 1=voltamperogramme after the consumption of 8 electrons;

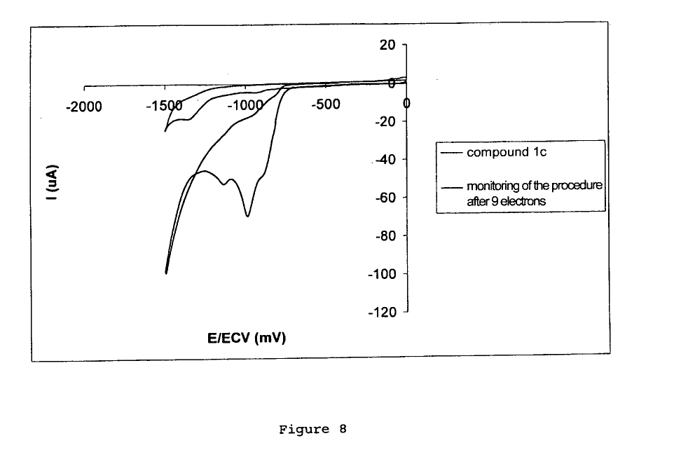

FIG. 8 shows a voltamperogramme of the preparative electrolysis of 6,6′-bis(4,6-dimethylpyridin-2-yl)-3,3′-bipyridazine (105);

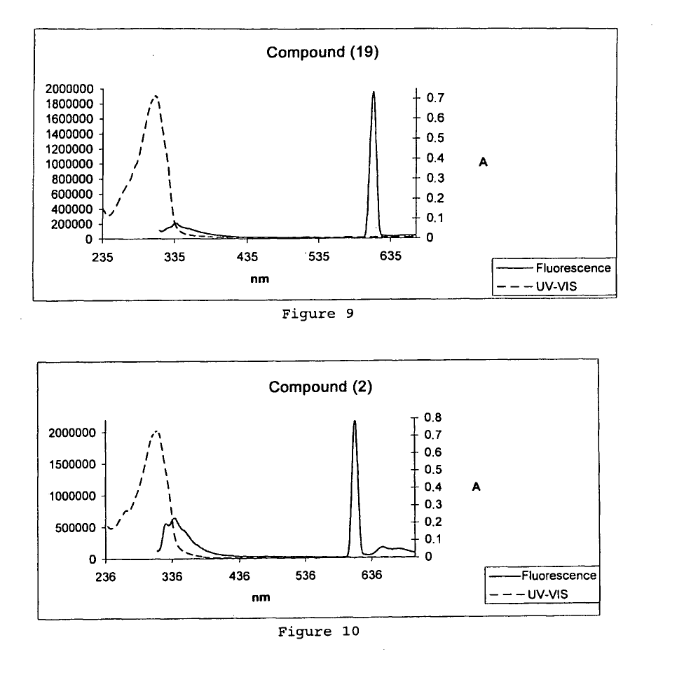

FIG. 9 shows the UV-visible and fluorescence absorption spectra of 6,6′-bis(pyridin-2-yl)-3,3′-bipyridazine (19);

FIG. 10 shows the UV-visible and fluorescence absorption spectra of 6,6′-bis(6-methylpyridin-2-yl)-3,3′-bipyridazine (2); and

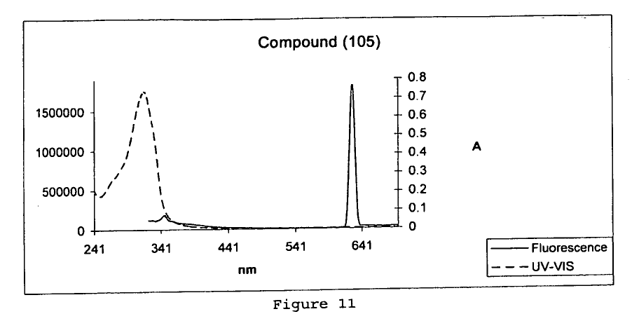

FIG. 11 shows the UV-visible and fluorescence absorption spectra of 6,6′-bis(4,6-dimethylpyridin-2-yl)-3,3′-bipyridazine (105).

### GENERAL CONDITIONS AND PROCEDURES RELATING TO THE EXPERIMENTAL PART

### Nuclear Magnetic Resonance (NMR)

The ¹H and ¹³C NMR spectra were recorded using a Bruker Avance 300 spectrometer. The irradiation frequencies were 300 MHz and 75.5 MHz, respectively, the chemical displacements are given in parts per million (ppm) with tetramethylsilane as internal standard. The coupling constants are given in Hertz (Hz) and the multiplicity of the signals is described as follows: s (singlet), brs (broad singlet), d (doublet), dd (doublet of doublets), t (triplet), q (quadruplet), m (multiplet).

### UV and Fluorescence Analyses

The UV-visible absorption spectra were recorded using a Shimadzu UV-2401PC spectrometer. The fluorescence spectra were recorded using a Fluoromax SPEX fluorimeter. All the spectra recorded using the devices listed above were performed in a UV-visible quartz cell (1 cm).

### Gas-Phase Chromatography (GC)

The chromatograms were recorded using a HP 6890 device fitted with a JW 1701 column (30 m×0.25 mm, stationary phase: cyanopropyl-phenyl-methylsilane), a flame ionisation detector, and nitrogen as vector gas (flow=1.3 mL/min). The temperature of the oven was programmed as follows: 1 minute at 80° C., then 12° C. per minute up to 280° C.

### Thin-Layer Chromatography

All reactions were followed by a thin-layer chromatography (Kieselgel 60F₂₅₄ Merck on an aluminium sheet). The plates were revealed by UV light or Mohr test (10% FeSO₄ in water).

### Mass Spectroscopy (MS)

The mass spectra were recorded using a Thermoelectron DSQ device by electronic impact (EI) (70 eV), chemical ionisation (CI) (ammoniac), direct introduction or GC-MS coupling.

### Solvents

All the solvents used were purchased in a pure form for the synthesis. The tetrahydrofuran (THF) was freshly distilled on sodium/benzophenone under argon. The dichloromethane (DCM) and N,N-dimethylformamide (DMF) were freshly distilled on calcium hydride under argon. The toluene was freshly distilled on sodium under argon.

### Procedure A: General Procedure of Ulmann-Type Homocoupling

The tetrabutylammonium bromide, the powder activated zinc and the nickel (II) dibromobistriphenylphosphine are added to a round-bottom flask. The mixture is dried under vacuum and placed under argon. The freshly distilled and degassed DMF is cannulated into the medium. The solution is stirred at room temperature until a homogeneous solution is obtained. The halopyridazine is solubilised in the DMF that has been freshly distilled, degassed and cannulated into the reaction medium. The solution is stirred for 15 hours at 55° C. The blackish solution is cooled to room temperature, treated with ammonia (25 N) and extracted with DCM. After drying of the organic phase over Na₂SO₄ and evaporation of the solvent under reduced pressure, the residue is purified.

### Procedure B: General Procedure of Acid Hydrolysis

In a round-bottom flask fitted with a condenser, methoxypyridazine and a 33% HBr solution in acetic acid are stirred for 48 hours at 60° C. The solution is then cooled and concentrated under vacuum. The precipitate is filtered and washed with acetone. The greyish solid is suspended in water. The solution is refluxed and neutralised with a 1M NaOH solution. The precipitate is filtered, washed with water, and dried under vacuum.

### Procedure C: General Chlorination Procedure

POCl₃ and pyridazinone are heated to reflux for 18 hours in a round-bottom flask fitted with a condenser. Upon return to room temperature, the excess is distilled off under vacuum and the residue is hydrolysed with ice. The solution is then neutralised by addition of 1M soda and extracted with dichloromethane. The organic phase is dried over Na₂SO₄ and concentrated under reduced pressure.

### Procedure D: General Procedure of Stille Coupling

The previously dried reagents are added to a round-bottom flask fitted with a condenser under argon (haloaryle, stannylpyridine, palladium catalyst), and the freshly distilled and degassed solvent is cannulated into the reaction medium. The solution is heated and stirred until the starting product has completely disappeared. Upon return to room temperature, the solvent is evaporated under reduced pressure, and the residue is taken up in DCM. The solution is then filtered through Celite and washed with DCM. The organic phase is then sequentially washed with concentrated ammonia (25 N) and a KF saturated solution. The organic phase is dried over Na₂SO₄ and concentrated under reduced pressure.

### Procedure E: General Procedure of Negishi Coupling

A bromopyridine solution (1.6 eq.) in the freshly distilled and degassed THF is cooled to −78° C. in a three-neck round-bottom flask fitted with a condenser. The butyllithium (2.5 M in hexane, 1.6 eq.) is added gently and the reaction medium is stirred for 30 minutes at −78° C. The zinc chloride solution (previously sublimated, 1.6 eq.) in the degassed THF is cannulated at −78° C. into the reaction medium. The solution is stirred at room temperature for 30 minutes, then a solution of tetrakis(triphenylphosphine) palladium (0) (0.1 eq.) and halopyridazine (1 eq.) in the THF is cannulated into the reaction medium. The solution is stirred for 48 hours at a temperature that depends on the substrate. The medium is treated with a NaHCO₃ saturated solution. The solution is filtered through Celite and sequentially washed with DCM and with concentrated ammonia (25 N). The organic phase is dried over Na₂SO₄ and concentrated under reduced pressure.

### Procedure F: General Procedure of Electrochemical Ring Contraction

The compound to be reduced is dissolved either in a three-solvent system (THF/acetic buffer/CH₃CN: 5/4/1) or in a 0.5M H₂SO₄ solution, and placed in the anode compartment of the electrochemical cell. An identical solvent system is placed in the cathode compartment and the appropriate voltage is applied until 8 electrons have passed. If necessary, the organic phase is evaporated under vacuum. The aqueous phase is then treated with a Na₂CO₃ saturated solution until an alkaline pH is achieved. The medium is extracted with DCM, and the organic phase is dried over Na₂SO₄, filtered and concentrated under vacuum. The residue is purified by silica column chromatography (EP/AcOEt: the ratio depends on the compounds).

### Example 1

### Synthesis of 6,6′-bis(pyridin-2-yl)-3,3′-bipyridazine 19, 6,6′-bis(5-methylpyridin-2-yl)-3,3′-bipyridazine 2, and 6′6′-dipicolin-4,4′-dimethyl-2-yl-[3,3′]bipyridazine (105)

### 1. Synthesis of 6,6′-bis(pyridin-2-yl)-3,3′-bipyridazine 19

A first traditional route for obtaining 6,6′-bis(pyridin-2-yl)-3,3′-bipyridazine 19 was considered according to the following retrosynthesis analysis (Retrosynthesis 1), that is inspired from the strategy developed by J. M. Lehn (Baxter, Lehn et al., 2000).

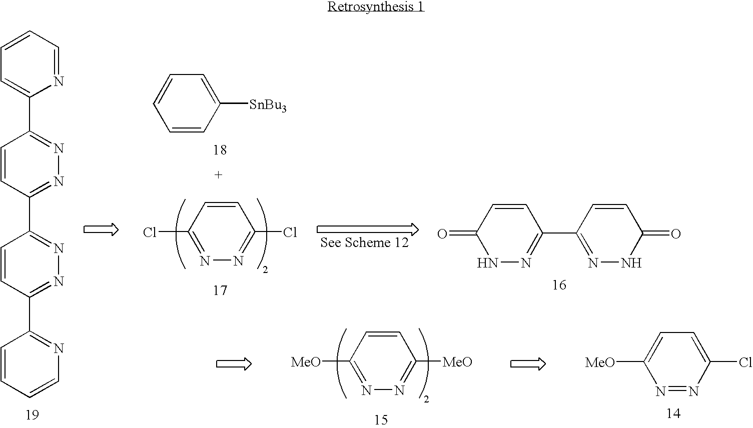

Different synthetic routes have been considered to obtain bipyridazine (19).

### Route 1:

Bipyridazine (15) is achieved through the homocoupling of 3-chloro-6-methoxypyridazine (14) in the presence of a catalyst: nickel (II) dibromobistriphenylphosphine with a yield of 36%. Bipyridazinone (16) is obtained after acid hydrolysis with a yield of 98%. Compound (16) is then chlorinated and placed in the presence of stannylpyridine (18) under Stille-coupling conditions to give the expected product (19).

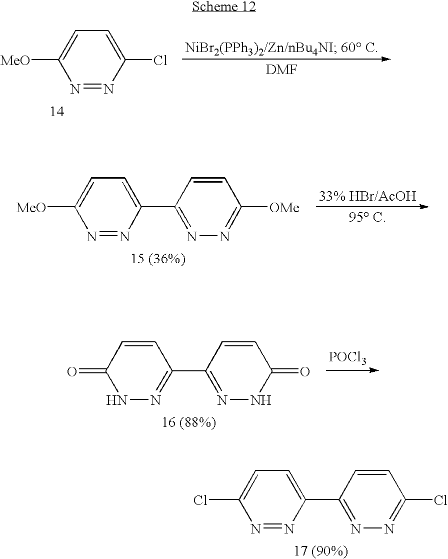

The bipyridazine unit introduced in the first step has complexation properties that can be potentially troublesome during extractions; a second synthetic route has therefore been considered. The aim of the second synthetic route is to introduce this unit during the final step.

### Route 2:

Stannylpyridine (18) is placed in the presence of 3-chloro-6-methoxypyridazine (14) under Stille-coupling conditions. Methoxypyridazine (102) is obtained with a yield of 77%. After acid hydrolysis and chlorination, the pyridazinone (7) and chloropyridazine (8a) are obtained with yields of 40% and 77%, respectively. Bipyridazine (19) is obtained through the homocoupling of 3-chloro-6-pyridylpyridazine (8a) in the presence of nickel (II) dibromobistriphenylphosphine, with a yield of 12%.

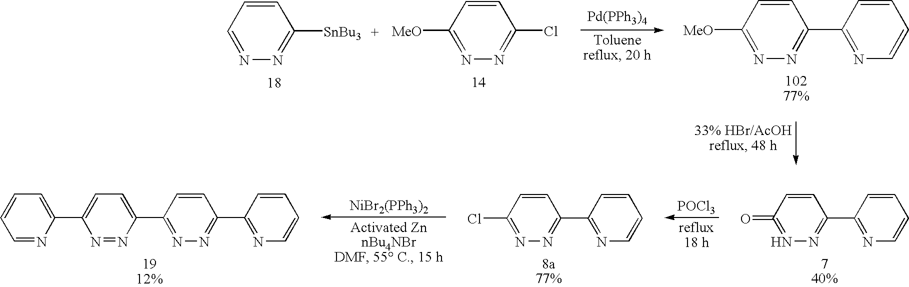

A third synthetic route has been developed in order to avoid the use of tin, and to reduce the number of steps.

### Route 3:

This synthetic route uses the Negishi coupling of an organozinc derivative with a halogenated pyridazine. The zinc pyridine is formed in situ from 2-bromopyridine in the presence of butyllithium and zinc chloride. A solution of 3-chloro-6-iodopyridazine (103) and tetrakis(triphenylphosphine) palladium (0) is then cannulated to give a compound (8a) with a yield of 62% (initial step). Bipyridazine (19) is achieved through homocoupling.

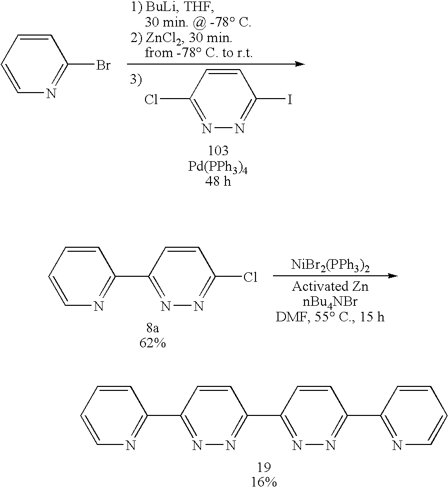

A study of the first step was undertaken in order to optimise the Negishi coupling process. Thanks to this study, yields increased from 14% to 62%, by using 3-chloro-6-iodo-pyridazine (103) instead of 3,6-dichloropyridazine at room temperature. The reaction only provides the monosubstitution product; the disubstitution product only appears upon heating, as a minor product.

| | Temperature | | | | |
|---|---|---|---|---|---|
| | −78° C.-r.t. | −78° C.-60° C. | −78° C.-r.t. | −78° C.-r.t. | −78° C.-60° C. |
| Reaction time | 24 hrs | 24 hrs | 24 hrs | 24 hrs | 24 hrs |
| 2-bromopyridine | 1.6 eq. | 0.9 eq. | 1.6 eq. | 1.6 eq. | 1.6 eq. |
| nBuLi | 1.6 eq. | 0.9 eq. | 1.6 eq. | 1.6 eq. | 1.6 eq. |
| ZnCl₂ | 1.6 eq. | 0.9 eq. | 1.6 eq. | 1.6 eq. | 1.6 eq. |
| Dichloropyridazine | 1 eq. | 1 eq. | 1 eq. | — | — |
| Pd(PPh₃)₄ | 0.05 eq. | 0.05 eq. | 0.10 eq. | 0.05 eq. | 0.05 eq. |
| 3-chloro-6- | — | — | — | 1 eq. | 1 eq. |
| iodopyridazine | | | | | |
| Yield | 18% | 14% | 48% | 62% | |
| Product mass | 204 mg | 180 mg | 364 mg | 464 mg | Mixture of |
| obtained | | | | | mono- and |
| | | | | | disubstituted |
| | | | | | products not |
| | | | | | separable by |
| | | | | | silica gel |
| | | | | | chromatography |

The nickel used in the homocoupling reaction may render molecule extraction difficult in the last step. A fourth route has therefore been developed.

### Route 4:

This synthetic route is nearly identical to Route 1, with Stille coupling being replaced by Negishi coupling so as to avoid the presence of traces of tin in the final compound.

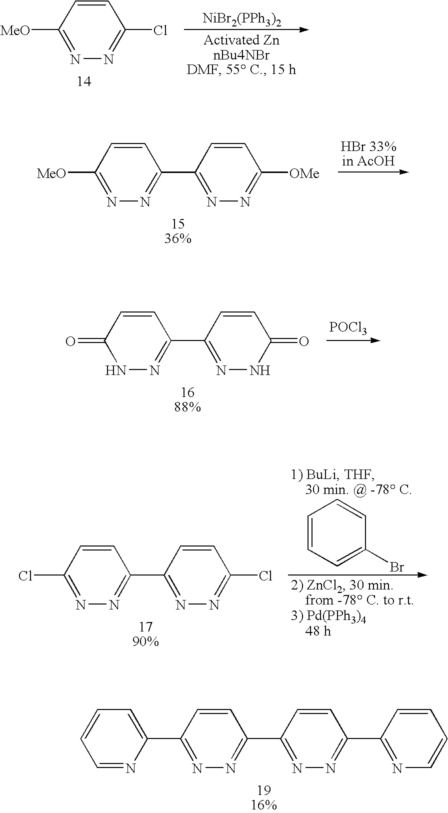

### 2. Synthesis of 6,6′-bis(5-methylpyridin-2-yl)-3,3′-bipyridazine 2 (also called 6,6′-dipicolin-2-yl-[3,3′]bipyridazine)

### Route 1:

The procedure described in Scheme 12 has also been reproduced for the preparation of 6,6′-bis(6-methylpyridin-2-yl)-3,3′-bipyridazine 2, which was obtained with a yield of 37%. The Stille-coupling reaction was carried out in this case from 6,6′-dichloro-3,3′-bipyridazine 17, in the presence of 6-methyl-3-tributylstannylpyridine 22 (Scheme 9)

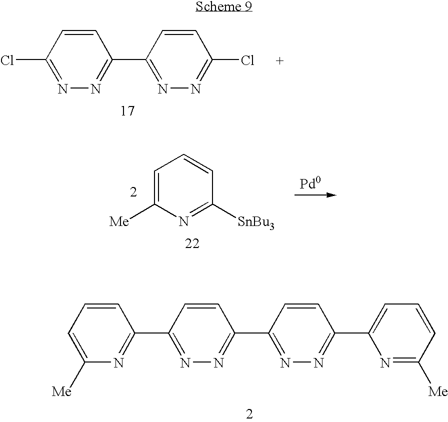

### Route 2:

This synthetic route corresponds to Route 4 described above.

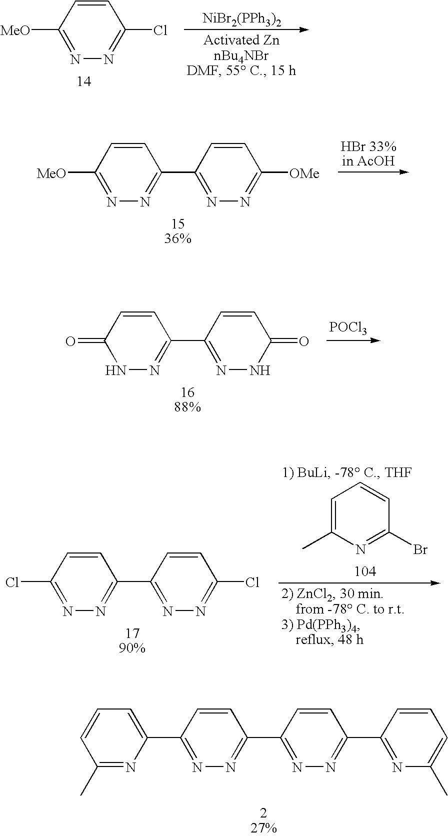

### 3. Synthesis of 6,6′-dipicolin-4,4′-dimethyl-2-yl-[3,3′]bipyridazine (105)

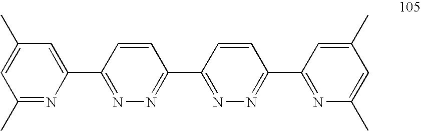

The synthetic route used also corresponds to Route 4 in Item 1. The addition of a further methyl substituent to the 6-methylpyridine group increases the solubility of the compound and hence facilitates the electrochemical ring regression.

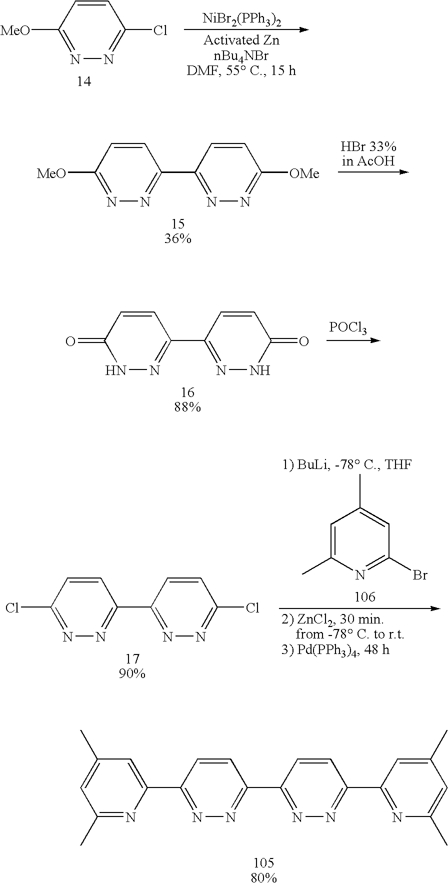

### Example 2

### Synthesis of 2,2,6,6′-bis(1-ethoxyvinyl)-3,3′-bipyridazine 24 via 6,6′-dichloro-3,3′-bipyridazine 17

Starting from 6,6′-dichloro-3,3′-bipyridazine 17, many functional modifications are possible, among which access to 6′-bis(1-ethoxyvinyl)-3,3′-bipyridazine 24, with a yield of 68% in the presence of tributyl-(1-ethoxyvinyl) tin and trans-bis(triphenylphosphine)palladium (II) [Pd(PPh₃)₂Cl₂], in DMF at 80° C. (Scheme 10).

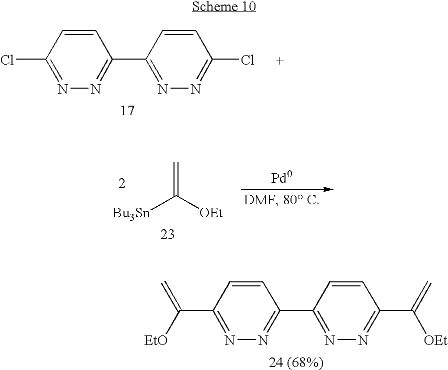

This molecule has a strong cytotoxic potential on KB cancerous cells, with an IC₅₀ of 0.3 μg/mL, and also affects the tonB protein that is involved in the process of iron transfer for bacterial growth.

### Example 3

### Electrochemical Synthesis of Linear Oligopyridazine Ligands

The sacrificial-anode method (J. Chaussard, J.-C. Folest, J.-Y. Nédélec, J. Périchon, S. Sibille, M. Troupel, Synthesis, 1990, 369-381) has made aromatic and heteroaromatic halide couplings possible.

### 1. Description of the Electrochemical Method Employed

The coupling of aromatic halides is possible thanks to an indirect electrolysis process catalysed by nickel complexes. The method employed is the sacrificial-anode method. The precursor of the catalyst is introduced in the form of nickel salts or by oxidation of a metal bar containing nickel (stainless steel or Fe/Ni 64/36 steel)

The material used is as follows:

The electrochemical cell is made of a glass wall that is terminated, in the bottom part, by a thread into which is screwed a bakelite base (black, SVL40) with an intermediary sealing ring. In the top part, four inlets of SVL15 type are arranged around a central SVL22 inlet to which a metal bar can be adapted as an anode. The cathode, made of nickel foam (40 cm²) is placed concentrically around the anode. Stirring of the solvent inside the cell is achieved using a magnetic stirrer bar. The aim of the various side inlets is to provide connection of the cathode using a stainless steel wire, and the inlet and outlet of a gas such as argon which ensures an inert atmosphere inside the electrochemical cell. Through the fourth inlet, samples can be taken from or reagents can be added to the reaction medium during electrolysis. If necessary, one of the inlets may be used to introduce a reference electrode in order to measure the evolution of the potential of the cell during the reaction.

The cell is placed in a magnetically stirred oil bath that makes heating possible, if necessary. DMF is the solvent employed in the process. The medium is rendered conductive by the introduction of support electrolytes such as quaternary ammonium salts. The power supply of the cell is achieved by means of a stabilised power supply thanks to which work is possible under an amperostatic regime of 10-300 mA.

The two reactions involved in the electrolysis are to occur simultaneously. The reaction at the cathode is a reduction of the species that is the most easily reduced and which is, in this case, the catalyst precursor (nickel II salts). Nickel (II) is thus reduced into nickel (0), stabilised by the ligands contained in the medium (pyridine or bipyridine). The counter-reaction at the anode is the oxidation of the metal bar that is made of iron or iron/nickel alloy with a 64/36 composition. The metal salts generated in the medium hence participate in the proper functioning of the reaction. The process involved around the nickel (0) is shown in Schemes 1 and 2, according to high (Scheme 1) or low (Scheme 2) imposed amperage.

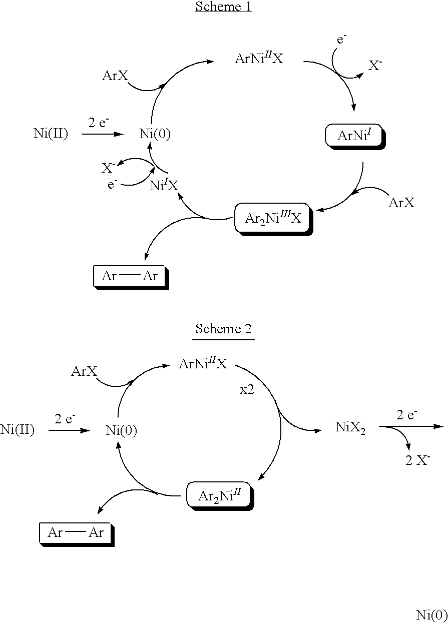

If the anode is made of iron, the precursor of the catalyst is the NiBr₂Bipy complex (10%) used, and in the case of an Fe/Ni (64/36) anode, the precursor of the catalyst is NiBr₂ (5-10%), and the ligand used as a co-solvent is pyridine.

### 2. Electrochemical Homocoupling of a Pyridizane Halide

6,6′-dimethoxy-3,3′-bipyridazine 15 is the key intermediate of the synthesis of 6,6′-bis-substituted 3,3′-bipyridazines. An original, simple and efficient synthesis of this intermediate achieved electrochemically has been devised. This synthesis uses the sacrificial-anode method described above and involves the homocoupling of 3-chloro-6-methoxypyridazine 14 through catalysis by nickel complexes (Scheme 1′)

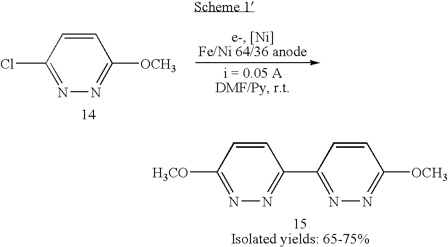

The material used is that described in paragraph 1. The anode is a bar of Fe/Ni (64/36) and the cathode is made of nickel foam (Goodfellow provider). The solvent is a DMF/pyridine 50/50 mixture and the support electrolyte is made of an NBu₄Br/NBu₄I 1/1 mixture. The reaction is conducted at room temperature in an argon atmosphere. The pre-electrolysis in the presence of dibromoethane (300 μL) is carried out during 15 min with an amperage of 0.1 A and in the absence of nickel (NiBr₂, ×H₂O, 10%) and of the reagent (3-chloro-6-methoxypyridazine). The latter are then added and the electrolysis is continued with an amperage of 0.05 A. The evolution of the reaction is followed by a CG analysis consisting of taking samples taken from the reaction medium and hydrolysing these samples (aqueous solution saturated with EDTA/CH₂Cl₂); this is continued until the aryl halide has completely disappeared (time: approximately 15-19 hours). The solvent is evaporated under reduced pressure. The residue is taken up in a mixture (aqueous solution saturated with EDTA and dichloromethane) and submitted to magnetic stirring for an hour. The organic phase is separated from the aqueous phase and the latter is extracted with CH₂Cl₂ (4 times 100 mL). The assembled organic phases are dried over anhydrous sodium sulfate, filtered and concentrated under reduced pressure. The obtained residue is purified by chromatography on neutral aluminium oxide (elution 100% CH₂Cl₂).

### Experiment Data:

6,6′-dimethoxy-3,3′-bipyridazine 1, CASE RN [24049-46-5]: White crystals; Obtained mass=695 mg; Yield=64; (aluminium gel purification, eluting: dichloromethane 100%). Melting point: 238-239° C. (litt.: 237-238° C.)

¹H RMN (CDCl₃, 300 MHz, δ ppm): 8.60 (d, 2H, J=9.3 Hz); 7.11 (d, 2H, J=9.3 Hz), 4.19 (s, 6H, OCH₃).

¹³C RMN (CDCl₃, 75 MHz, δ ppm): 165.47; 152.47; 127.41; 118.21; 55.07.

SM (EI) M/Z (%): 219 (13), 218 (100), 217 (57), 189 (32), 175 (33), 147 (31), 119 (12).

Similarly, the preparation of 6,6′-dichloro-3,3′-bipyridazine 17 can be achieved by electrochemical coupling of 3,6-dichloropyridazine 1′ according to the following reaction scheme (Scheme 2′):

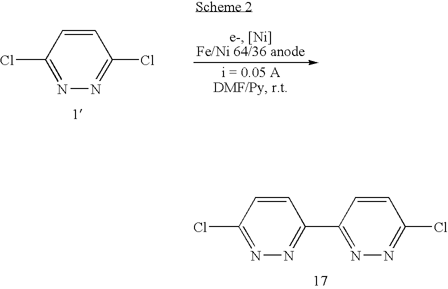

### 3. Electrochemical Heterocoupling of a Pyridazine Halide

The reaction involved is as follows (Scheme 4′):

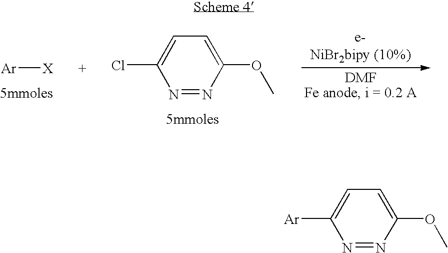

Unlike homocouplings, heterocouplings were conducted with an amperage of 0.2 A. The precursor of the catalyst is a NiBr₂bipy complex added in catalytic quantities (10%). The anode is made of iron (XC₁₀, 0.1% carbon). The solvent is the DMF.

The results are listed in the table below (Table 1).

TABLE 1
Heterocoupling of aromatic halides with 3-chloro-6-methoxypyridazine

| | | | Isolated yield |
|---|---|---|---|
| Item | ArX | Product | (%) |
| 1 | 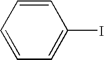 | 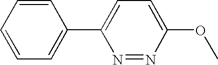 | 60 |
| 2 | 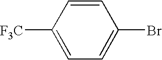 | 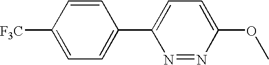 | 62 |
| 3 | 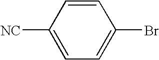 | 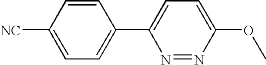 | 58 |
| 4 | 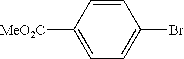 | 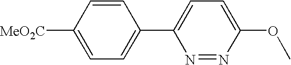 | 70* |
| 5 | 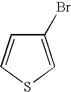 | 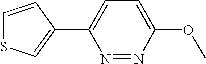 | 56* |
| 6 | 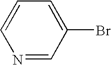 | 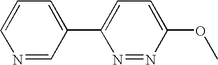 | 33 |

*Amperage: 0.05 A

The obtained yields are around 60%, except for 3-bromopyridine (item 6) where a decrease is observed (33%) For methyl parabromobenzoate (item 4) and 3-bromothiophen (item 5), the reactions were conducted at 0.05 A.

According to the retrosynthesis scheme shown hereunder (Retrosynthesis 1′), it is therefore possible to consider two approaches to bipyridazines by electrochemical couplings.

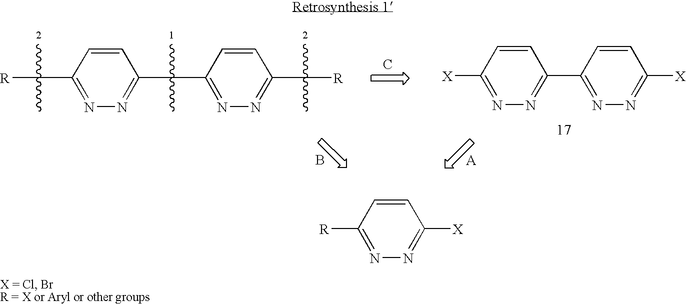

For the retrosynthesis Route A, a coupling that gives 6,6′-dichloro-3,3′-bipyridazine 17 through the homocoupling of 3,6-dichloropyridazine 1′ is considered (see above).

Starting from 17, many bipyridazine structures are achievable chemically or electrochemically, for which. R is a variable.

As regards the retrosynthesis Route B, the synthesis of 6,6′-bis(pyridin-2-yl)-3,3′-bipyridazines 19 and 2 is performed in two electrochemical steps described in Scheme 7′ via the intermediate 2′.

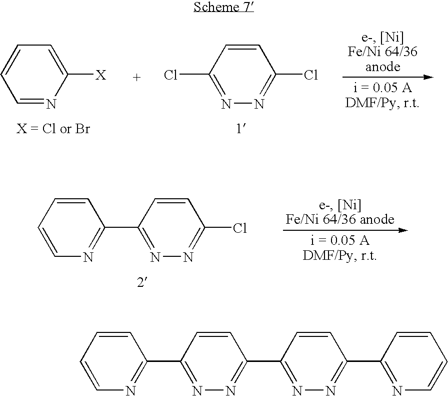

Furthermore, with this route, asymmetric bipyridazine analogues can be obtained, in which R and R′ are variables (Scheme 8′).

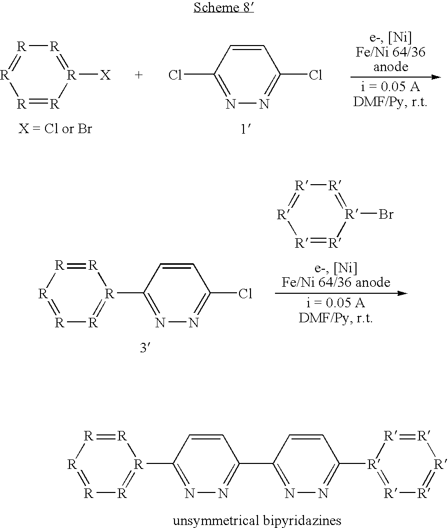

### Example 4

### Electrochemical Synthesis of Bipyrrole Sequences

Some bipyridazine structures were submitted to electrochemical ring-regression conditions. The electroreduction experiment was carried out in a sulphuric acid environment because 3,3′-bipyridazines 2 and 19 are not very soluble in the acetic buffer. The conditions were as follows: vitreous carbon electrode, V=100 mV/s. The voltamperogramme of 6,6′-bis(6-methylpyridin-2-yl)-3,3′-bipyridazine 2 indicates clearly a very marked reduction wave with a potential of −0.5 V/ECS and a slight shoulder around −0.6 V/ECS (FIG. 1)

According to preliminary studies, it was possible to consider that the first wave corresponds to the potential of a 2-electron simultaneous reduction (per pyridazine ring) of the two symmetrical pyridazine groups of dimer 2 (total of 4 electrons). The formation of a bis-dihydropyridazine 68 intermediate could therefore be considered and its reduction into dipyrrole 71 would logically need another further 4 electrons at least per mole (2 electrons per dihydro ring) for a total of eight electrons per mole from bipyridazine 2 (Scheme 121)

The preparative electrolysis of this compound was therefore carried out in a sulphuric medium by applying a working potential −0.5 V/ECS during on hour (Q=210 C) then it is continued at a potential of −0.6 V/ECS until near full consumption of the precursor (Q=305 C). The monitoring of this preparative electrolysis was conducted by cyclic voltamperometric measures taken directly in the cathode compartment on the carbon electrode at different stages of the electrosynthesis (Scheme 1: —before electrolysis, —during electrolysis, at the end of the electrolysis). The diminution of the intensity of the reduction wave of the bipyridazine 2 compound on the different voltamperogrammes demonstrates clearly the reduction of the latter during the preparative electrolysis. The latter was stopped after a total disappearing of this wave (4.5 hrs into the experiment, during which at least 4 electrons have therefore been used).

At the end of the electrolysis, the consumption of Coulomb in this experiment only corresponds in reality to 4.78 electrons per mole of bipyridazine substrate (for a theoretical value of Qt=440C=8e⁻).

This 4-electron electroreduction (Scheme 121) according to this procedure mainly leads to the formation of 3-[5-(6-methylpyridin-2-yl)-pyrrol-2-yl]-6-(6-methylpyridin-2-yl)-pyridazine 70.

The selective reduction of a single ring out of the two is therefore possible. The mechanism involves either a first 4-electron reduction of the pyridazine dimer into bis-1,2-dihydropyridazines 68, or two successive 2-electron reduction steps via the intermediate 69. This hypothesis was at first suggested by the identification in the medium of the intermediate of 3-[6-(6-methylpyridin-2-yl)-dihydropyridazin-3-yl]-6-(6-methylpyridin-2-yl)-pyridazine 69 that results from a first 2-first electron reduction.

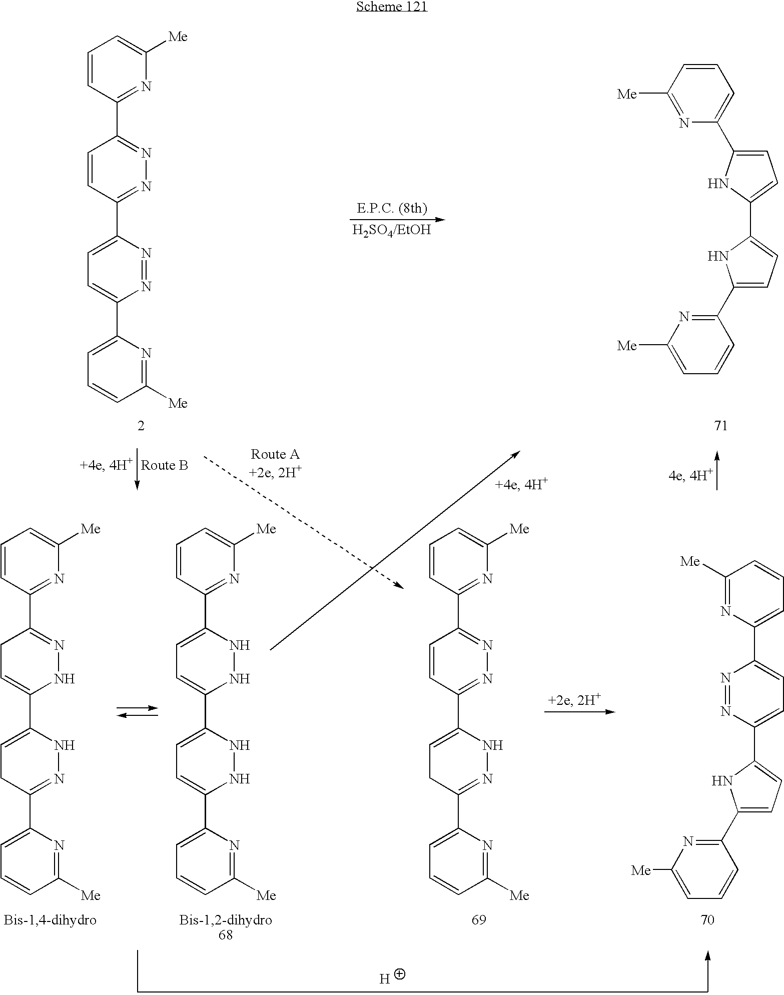

The identification of the two compounds was confirmed by their mass spectrum, m/z=[M−H] 341 for dihydro 69 and m/z=[M] 327 for the pyrrole 70 compound, respectively. The analysis of the ¹H RMN spectrum of the pyrrole 70 indicates the presence of NH at δ=11 ppm and two pyrrole protons at δ=6.77 and 6.78 ppm (³J=3.9 Hz). These can also be coupled with the NH of a pyrrole, with coupling constants of ⁴J of 2.4 Hz and −2.7 Hz, respectively.

This alternative also offers the possibility of obtaining alternate pyridazine-pyrrole systems.

The preferred mechanism seems to work towards the formation of bis-1,2-dihydropyridazine 68 (resulting from a 4-electron reduction) that is rearranged in pyrrole-pyridazine 70. Afterwards, the bipyrrole 71 results from a new 4-electron reduction of the remaining pyridazine.

According to the procedure developed for this electroreduction experiment, in which only 4.78 electrons per mole were used, the generation of bipyrrole 71, which requires 8 electrons per mole of bipyridazine 2, could not be optimal. Its presence was nevertheless identified under these conditions, but in low proportions. The ¹H RMN of the reaction results indicates clearly that the NH peak of the pyrroles occurs at =9.65 ppm for this symmetric molecule and that its mass spectrum complies with (m/z=[M+1] 313).

¹H RMN (300 MHz, CDCl₃) of 71: 9.65 (brs, 2H, NH); 7.45 (t, 2H, ³J=7.5 Hz, 2H4′ pyridinyl); 7.31 (d, 2H, ³J=7.5 Hz, 2H3′ pyridinyl); 7.31 (d, 2H, ³J=7.5 Hz, 2H5′ pyridinyl); 6.66 (m, 2H, 2H pyrrole); 6.41 (m, 2H, 2H pyrrole); 2.49 (s, 6H, CH3).

### Preparative Electrolysis of Bipyridazine 2, 8-Electron Reduction

The study was repeated by progressively moving the electroreduction potential towards more negative values of −0.7 V/ECS until the substrate has completely disappeared (or 8.15-electron consumption per mole of substrate). The main wave's disappearance was noted during the previous experiment, which coincided with the apparition of another wave with a potential of about −0.8 V/ECS (FIG. 1). It corresponds to the reduction of 3-[5-(6-methylpyridin-2-yl)-pyrrol-2-yl]-6-(6-methylpyridin-2-yl)-pyridazine 70 into the corresponding bipyrrole 71.

As in the previous electrolysis, the monitoring was achieved by cyclic voltamperometric measurements (FIG. 2).

In FIG. 2, it may be observed that after the passage of four electrons, there is still some starting product, as marked by the three first waves that are still present. The apparition of a second wave that is more cathodic in nature is typical of the reaction's final product.

The treatment, identical to the previous trial, also provides a mixture of reaction products, and the RMN analysis of the reaction results show many peaks. However, two major fractions were successfully isolated by chromatography: one containing 1,2,3,4-tetrahydropyridazinyl-pyrrole 72, and the other containing the expected bipyrrole 71 (Scheme 122).

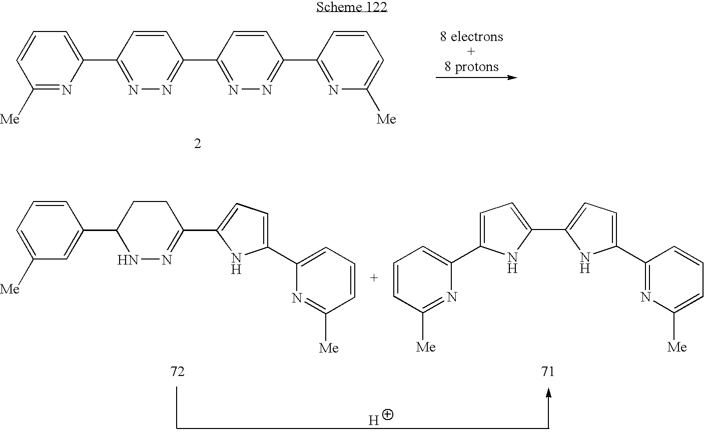

The presence of the tetrahydropyridazinepyrrole 72 reaction product enables us to conclude that the 8 electrons are used to reduce the pyridazine, as 72 represents the synthesis intermediate of bipyrrole 71.

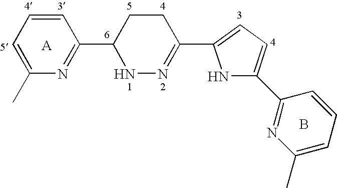

¹H RMN (400 MHz, CDCl₃) 72: 10.07 (brs, 1H, NH pyrrole); 7.62 (t, 1H, ³J=7.7 Hz, H4, pyridinylA); 7.41 (t, 1H, ³J=7.7 Hz, H4′ pyridinylB); 7.34 (d, 1H, ³J=7.7 Hz, H3, pyridinylB); 7.26 (d, 1H, ³J=7.7 Hz, H3′ pyridinylA); 7.09 (d, 1H, ³J=7.7 Hz, H5′ pyridinylA); 6.88 (d, 1H, ³J=7.7 Hz, H5′ pyridinylB); 6.64 (m, 1H, H4 pyrrole); 6.33 (m, 1H, H3 pyrrole); 6.02 (brs, 1H, NH tetrahydropyridazine); 4.30 (dd, 1H, ³J=9.9, 3.09, H6 tetrahydropyridazine); 2.70 (m, 1H, H4 tetrahydropyridazine); 2.65 (m, 1H, H4 tetrahydropyridazine); 6.64 (s, 6H, CH3); 2.54 (m, 1H, H5 tetrahydropyridazine); 2.16 (m, 1H, H5 tetrahydropyridazine);

¹³C RMN (400 MHz, CDCl₃) 72: 160.4 (C2′ pyridinylA); 158.2 (C6′ pyridinylA); 157.9 (C6, pyridinylB); 149.7 (C2′ pyridinylA); 138.5 (Cq pyrrole); 136.7 (C4′ pyridinylA); 136.5 (C4′ pyridinylB); 132.6 (Cq pyrrole); 122.2 (C3′ pyridinylA); 121.0 (C3, pyridinylB); 120.1 (C5′ pyridinylA); 117.5 (C5, pyridinylB); 108.2 (C4 pyrrole); 107.5 (C3 pyrrole); 57.5 (C6 tetrahydropyridazine); 26.0 (C5 tetrahydropyridazine); 24.6 (2 CH3); 22.1 (C4 tetrahydropyridazine).

However, an optimisation of the method can involve the potential that needs to be applied (up to 0.85 V/ECS) or simply letting the reaction proceed. The hypothesis is that at −0.7 V/ECS, dihydropyridazine 2 is reduced into 72 and that the rearrangement of pyrrole 71 in an acid medium simply requires a longer reaction time.

### Example 5

### Reduction of 6,6′-bis(pyridin-2-yl)-3,3′-bipyridazine by preparative electrolysis with controlled potential

The preparative electrolysis is carried out in a sulphuric acid medium (0.5 mol.L⁻¹)/ethanol (proportion: 0.5/0.5) in a cell with two compartments separated by fritted glass. In the anode compartment the anode and a stainless steel plate with a surface area of 15 cm² are placed. The cathode, a mercury layer with an area of 16 cm² and the reference electrode, the saturated calomel electrode, are placed inside the cathode compartment. The solvent volume in both compartments is of 90 mL. The substrate is introduced in the cathode compartment (194 mg, i.e. 5.7.10⁻⁴ mol) and the potential applied at the beginning of the electrolysis is −0.5 V/ECS (reduction potential for the substrate), the corresponding amperage is 35 mA. After 1 hour of electrolysis, it is continued at −0.6 V/ECS until the substrate has disappeared (4.5 hrs), the amperage at the end of the electrolysis is of 8 mA. The monitoring of the electrolysis is achieved by cyclic voltamperometric measurements taken on the carbon electrode (S=3.2 mm²) directly in the cathode compartment. The consumption of coulomb during the electrolysis is 305 C, or 5.57 electrons per mole of substrate. The reaction medium of the cathode compartment is evaporated to eliminate the ethanol, and neutralised with NaHCO₃. After extraction by dichloromethane, the organic phase is dried over Na₂SO₄ and evaporated. The monopyrrole compound 3-[5-(pyridin-2-yl)-pyrrol-2-yl]-6-(6-methylpyridin-2-yl)-pyridazine is purified by silica gel chromatography.

### 5,5′-bis(pyridin-2-yl)-2,2′-bipyrrole

In order to optimise the electrosynthesis of the bipyrrole, the electrolysis under the same operating conditions (solvent, electrodes), but with a progression of the electroreduction potential towards more negative values (E=−0.85 V/ECS), leads to the formation of the bipyrrole system.

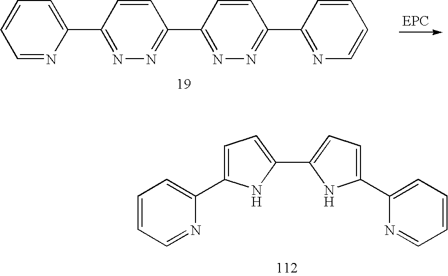

Solubilisation difficulties on compound 19 have prevented the electrochemical regression under the traditional conditions (EtOH/Acetic buffer); solubilisation in 0.5M sulphuric acid is the only possibility. The voltamperogrammes recorded in 0.5M sulphuric acid have highlighted three successive reduction waves at potentials of Ec=−0.363 V, −0.500 V and −0.673 V. Under similar conditions, the pyrrole compound shows a reduction wave that corresponds to the reduction of the pyrroles with a potential of E, =−0.818 V.

The preparative electrochemical electrolysis of bipyridazine (19) was carried out in a 0.5M H₂SO₄ medium. A potential of Et=−0.650 V/ECS was applied to the working electrode (mercury layer) until consumption of 8 electrons. The monitoring of the reaction was performed by cyclic voltamperometric measurement and TLC (see FIG. 7).

The bipyrrole 112 compound was obtained with a yield close to 10%, which was also the case for the monopyrrole 107 compound and the tetrahydropyridazine 108 compound. The low yield obtained for the bipyrrole is explained by its partial degradation in the concentrated H₂SO₄ medium and difficulties encountered with flash on silica gel chromatography purification.

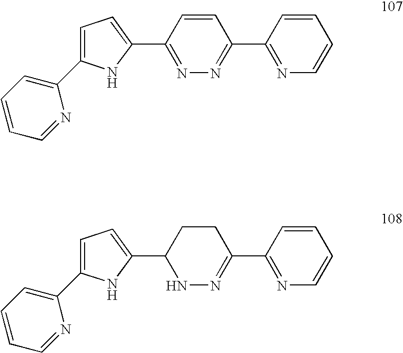

### Example 6

### Analysis of the Reduction of Polycyclic Monopyridazines

The analysis studies have demonstrated that the 3,6-bis(pyridin-2-yl)-pyridazines 80, 82, 83 and 85 are marked by similar voltamperogrammes to their 3,6-dicarbomethoxy-pyridazine homologues. Thus, the first four electron peaks are well defined and appear at potentials between −0.9 V and −1.1 V (FIG. 3, cyclic voltamperogramme of the solvent and precursors 80, 82, 83 and 85 in an acetic and ethanol medium, C=10⁻³ mol.L⁻¹, V=100 mV/s).

The voltamperogrammes of the precursors 4-carbomethoxy-3,6-bis(pyridin-2-yl)-pyridazines 84 and 3,6-bis(pyridin-4-yl)-pyridazine 81 are more complex, and several reduction waves occur as from more positive potentials (−0.7 V) (FIG. 4, cyclic voltamperogrammes of precursors 81 and 84 in an acetic+ethanol buffer medium, C=10⁻³ mol.L⁻¹, V=100 mV/s).

The values of the potentials and Ep peaks of the two first reduction peaks for these different precursors are listed in Table 4.

TABLE 4

| | Ep | Ep |
|---|---|---|
| Precursor | (peak I) | (peak II) |
| 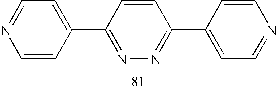 | −0.79 V | −0.91 V |
| 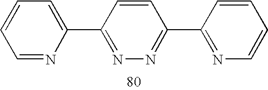 | −0.87 V | −1.06 V |
| 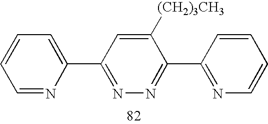 | −0.89 V | −1.08 V |
| 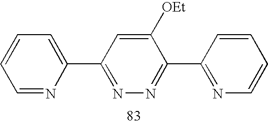 | −0.90 V | −1.02 V |
| 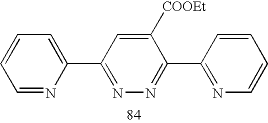 | −0.70 V | −1.11 V |
| 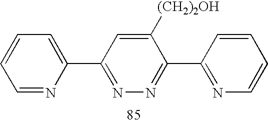 | −0.99 V | −1.18 V |

The determination of the number of electrons concerned by the reduction wave is achieved by cyclic voltamperometric measurements by adding an internal reference (Red/Ox couple with a known number of electrons) in the solution. The internal reference that was used is ferrocenemethanol (Fc); its oxidation peak potential is 0.28 V/ECS under the specific conditions of the experiment (Scheme 91)

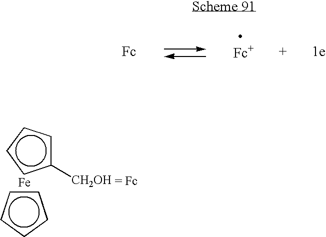

The results of the analysis confirm that the electroreduction of pyridinyl-pyridazines into pyrroles requires four electrons. The first 0.2-electron transfer generates dihydropyridazine intermediates (1,2- or 1,4-dihydropyridazines depending on their relative stability), and the second 2-electron transfer provokes the extrusion of a nitrogen atom by ammoniac release. The potential that is to be applied for all preparative electrolysis must therefore be at the level of the reduction waves that correspond to at least a 4-electron reduction.

### Example 7

### Preparative electrolysis of 3,6-bis(pyridinyl)-pyridazines

The preparative electrolysis of the various pyridazines (80-85) has been achieved by adopting a working potential that corresponds to the potential of the second reduction wave of the precursors, respectively. These were continued until total disappearance of the precursors and consumption of a quantity of charges (number of Coulombs) that corresponds to the minimal amount of electrons necessary to induce their arrangement into a pyrrole (four electrons per mole of the reduced substrate).

Starting from nearly the same initial concentration, the average amount of time for the entire disappearance of the precursors is between 5 and 6 hours. The pyrrole products 86, 87, 88, 89 and 90 were obtained with variable yields that range from 60 to 90% (Scheme 92, Table 5).

TABLE 5

Scheme 92

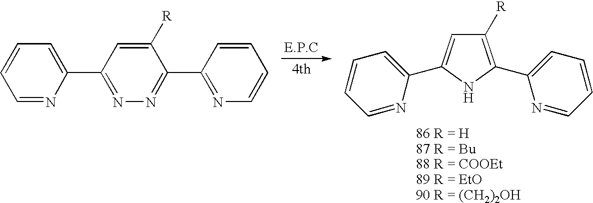

| | | | | | t | Yield | Chemical |
|---|---|---|---|---|---|---|---|
| Precursor | Ep(waveI) | Ep(waveII) | E_{imposed} | ne⁻* | (hrs) | (pyrroles) | yield*** |
| 80 | −0.87 V | −1.06 V | −1.00 V | 4.06 | 4 | 86 (82%) | 22% |
| 82 | −0.89 V | −1.08 V | −1.00 V | 4.53 | 6 | 87 (85%) | 18% |
| 83 | −0.90 V | −1.02 V | −1.00 V | 4.5 | 5.20 | 88 (75%) | 30% |
| 84 | −0.71 V | −1.12 V | −1.05 V | 5.66 | 6.25 | 89 (60%) | 32% |
| 85 | −0.99 V | −1.18 V | −1.10 V | 4.5 | 5.33 | 90 (92%) | 25% |

*number of electrons used/mole of reduced substrate
**Zn/AcOH

As these results demonstrate, in all cases, the regression of the pyridazine ring in an acetic buffer medium seems rather more efficient electrochemically than chemically (Zn/AcOH), as recommended in the literature.

These results are confirmed in the case of pyridin-4-ylpyridazine 81 where the transformation in pyrrole 91 is achieved with a yield of 85% instead of the 30% achievable chemically (Table 6).

TABLE 6

| | | | | | t | Yield | Chemical |
|---|---|---|---|---|---|---|---|
| Precursor | Ep (waveI) | Ep (waveII) | ^{E}imposed | ne⁻* | (hrs) | (pyrroles) | yield*** |
| 81 | −0.79 V | −0.91 V | −0.95 V | 3.77 | 4.6 | 91 (85%) | 30% |

*number of electrons used/mole of reduced substrate
** Zn/AcOH

The progress of the reaction by preparative electrolysis was controlled in each instance by cyclic voltamperometric measurements directly taken in the cathode compartment on the vitreous carbon electrode, as shown on the voltamperogrammes recorded during the electrolysis of the precursor. 82 (FIG. 5, cyclic voltamperogrammes during the preparative electrolysis of precursor 82, —before electrolysis, —during electrolysis, —at the end of the electrolysis, vitreous carbon electrode, V=100 mV/s))

The intensity decrease of the reduction wave (−1.08 V) of the precursor during the preparative electrolysis is correlated to the transformation during electrosynthesis. Furthermore, it disappears totally at the end of the electrolysis, which makes it possible to check the total consumption of the precursor.

At the end of the electrolysis, the voltamperogrammes were also checked (FIG. 6, voltamperogrammes of the different pyrrole derivatives at the end of the electrolysis in the cathode compartment, vitreous carbon electrode. They show the disappearance of the two reduction waves, which confirms the mechanical hypothesis that was suggested. The first wave is attributed to 2-electron reduction of the pyridazines into dihydropyridazines and the second wave corresponds to nitrogen extrusions which give pyrrole rings.

### Example 8

### Preparative electrolysis of 6,6′-dipicolin-4,4′-dimethyl-2-yl-[3,3′]bipyridazine (105)

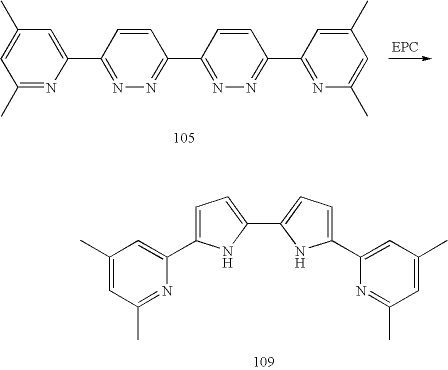

The solubility of bipyridazine has been enhanced by the addition of alkyl chains to the pyridine substituents (compound 105). It is therefore possible to solubilise bipyridazine 105 under milder conditions than the H₂SO₄ or acetic buffer media: a mixture of THF/Acetic buffer (pH=4.6)/Acetonitrile solvents. Les voltamperogrammes recorded under these conditions have highlighted three successive reduction waves at potentials of E, =−0.92 V, −1.03 V and −1.16 V (see FIG. 8).

The preparative electrolysis of bipyridazine (105) at ET=−1.05 eV has made isolation of bipyrrole (109) possible with a 35% yield (see table below, item 1). The results confirm the hypothesis that the low yield achieved during the preparative electrolysis of bipyridazine (105) was due to the degradation of the bipyrrole compound in the sulphuric acid.

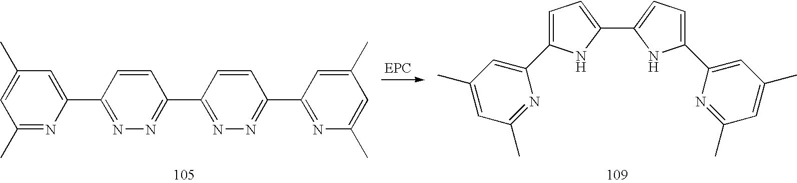

| Medium | Applied Potential | | Results |
|---|---|---|---|
| THF/acetic buffer | 1 | Et = −1.05 V, 10.3 e | Yield = 35% |
| pH = 4.6/Acetonitrile: | 2 | Et = −1.15 V, 8 e | Mixture of 2 compounds non separated, Among which is trace bipyrrole |
| 50/45/5 | | Stops for voltage | (109) |
| Polarograph | 3 | Et = −1.15 V, 10.1 e | pyrrole-pyridazine (110) 20% pyrrole-tetrahydropyridazine (111) 20% |
| measurements | | Without stopping + | |
| Ec = −0.92 V, −1.03 V, | | 1 hr break at the | |
| −1.16 V | | end before extraction | |
| | 4 | Et = −0.9 V, 3.78 e, | Rough RMN degradation products: waste |
| | | break 40 min | |
| | | Et = −0.97 V, 4 e | |
| | | break 40 min | |
| | | Et = −1.15 V, 3.1 e, | |
| | | left to run all night | |

Several experiments were conducted at different potentials and under different conditions (above table, entries 2-4). Only one modification (item 3) turned out to be interesting as it made isolation of two intermediates possible: monopyrrole-pyridazine (110) and monopyrroletetrahydropyridazine (111) with yields of 20% respectively.

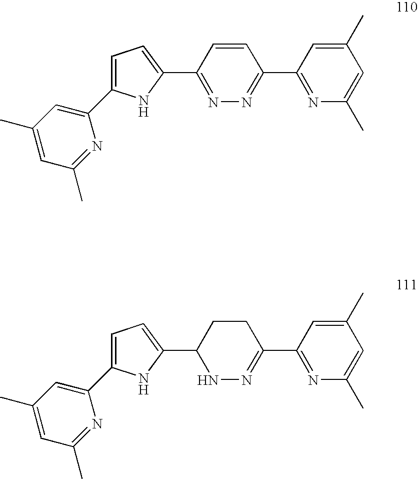

### Example 9

### Preparation of 3-(2-bromopyridin-6-yl)-6-(pyridin-2-yl)-pyridazine 43

The chloropyridazine 8a derivative treated in the presence of distributylstannyl and tetrakistriphenylphosphine palladium provides a mixture of products 39, 40 and 41.

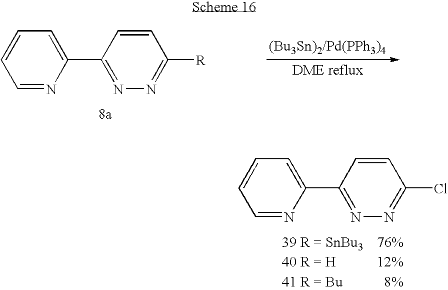

The monosubstituted intermediate, 3-(2-bromopyridin-6-yl)-6-(pyridin-2-yl)-pyridazine 43, is mainly isolated with a yield of 72%, starting from an equimolar mixture of stannylpyridazine 39 and of dibromopyridine 42 in the presence of tetrakistriphenylphosphine palladium in toluene (Scheme 17).

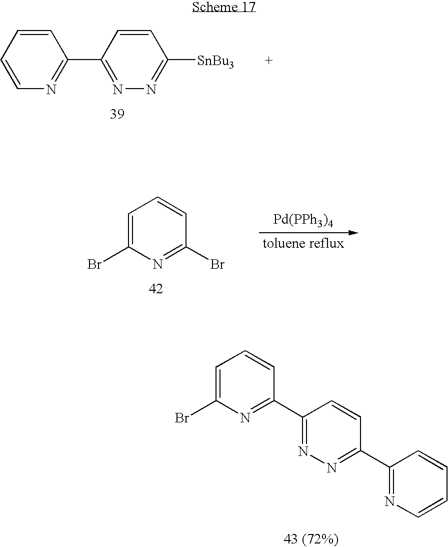

### Example 10

### Synthesis of 3-(2-carboxypyridin-6-yl)-6-(pyridin-2-yl)-pyridazine 45

In order to introduce mono functionalisation, chloro(pyridyl)-pyridazine 8a was selected as a precursor in the Stille-coupling reaction to react with methylated stannylpyridine 22 (Scheme 19), in the presence of equimolar tetrakistriphenylphosphine palladium, giving exclusively the coupling product 44 with a yield of 90%.

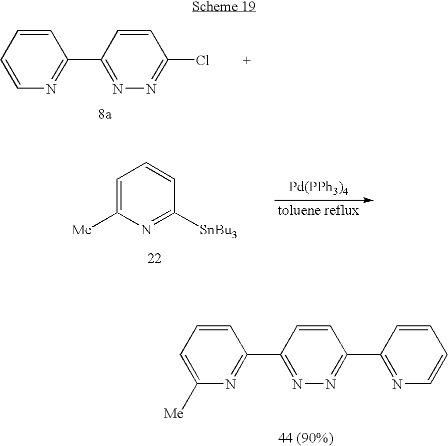

It is important to note that no trace of bispyridine homocoupling product is observed in the reaction mixture.

The oxidation aromatic methyl in α of pyridine nitrogen has been performed with selenium dioxide, at 150° C. in the o-dichlorobenzene, and 3-(2-carboxypyridin-6-yl)-6-(pyridin-2-yl)-pyridazine 45 was obtained with a yield of 74% (Scheme 20).

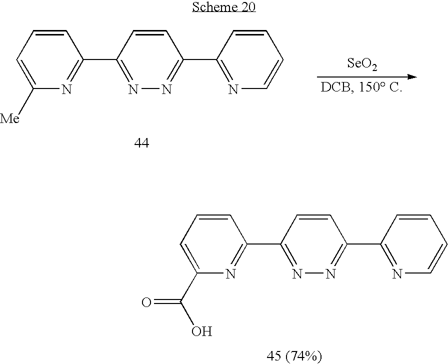

### Example 11

### Synthesis of the bis-tridentate diacid: 3,6-bis-(2-carboxylpyridin-6-yl)-pyridazine 48

According to the same principle, the pyridazine diacid ligand 48 can be obtained by oxidation of bis(dimethylpyridyl)-pyridazine 47 (Scheme 21) This is prepared via a double Stille coupling of 6-methyl-2-tributhylstannylpyridine 22 with 3,6-dichloropyridazine 46 in the presence of tetrakistriphenylphosphine palladium. In this case, a slight excess of stannylpyridine (3 eq.) was used. Under the same oxidation conditions as previously defined, the 3,6-bis(2-carboxylpyridin-6-yl)-pyridazine acid 48 is isolated with a yield of 68%.

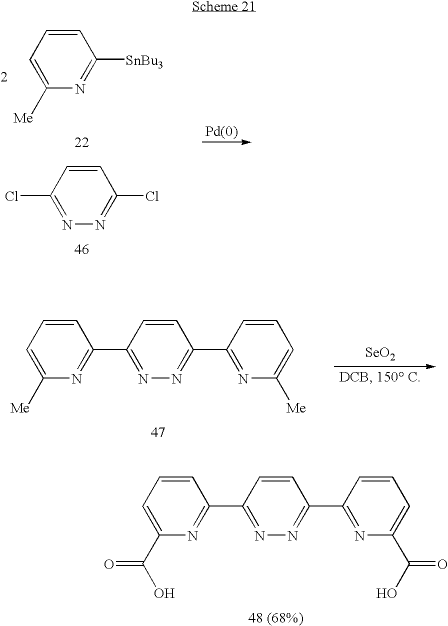

The pyridazine diacid 48 obtained is a bis-tridentate ligand: N-donor (pyridine and pyridazine) and O-donors (diacid). In this Example, the presence of two adjacent nitrogen atoms in the pyridazine ring contributes to generate two distinct coordination sites.

### Example 12

### 6,6′-bis(6-methylpyridin-2-yl)-3,3′-bipyridazine 2

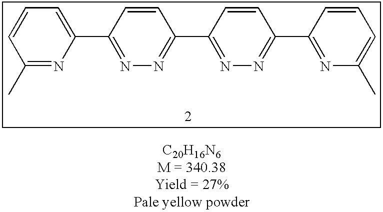 [0315] This compound is synthesised according to

Procedure E from 2-bromo-6-methylpyridine (104) and from 6,6′-dichloro-[3,3′]bipyridazine (17). The residue is heated and recrystallised in the AcOEt, thus giving the expected result with a yield of 27%.

¹H RMN (CDCl₃) δ (ppm): 7.45 (m, 2H, H_{pyridine}) 7.94 (dt, J=7.2, 1.8, 2H, H_{pyridine}) 8.79 (m, 6H, 2H_{pyridazine}, 4H_{pyridine}) 8.80 (d, J=9.0, 2H, H_{pyridazine}), 9.01 (d, J=9.0, 2H, H_{pyridazine}).

¹³C RMN (CDCl₃) δ (ppm): 121.8, 125.0, 125.4, 125.5, 137.3, 149.6, 153.1, 156.0, 159.2.

MS, m/z (I %): 340 (M⁺, 100%), 312 (M⁺-N₂, 49%).

UV/Fluorescence (DCM): see FIG. 10.

### Example 13

### 6-(pyridin-2-yl)-2H-pyridazin-3-one 7

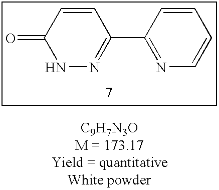

This compound is separated according to Procedure B with 3-methoxy-6-(pyridin-2-yl)-pyridazine (102) (0.29 g, 1.55 mmol) and a HBr solution (33% in acetic acid, 1.2 mL). The product was obtained in the form of white powder in a quantitative manner.

¹H RMN (DMSO-d₆) δ (ppm): 7.01 (d, J=9.6, 1H, H_{pyridazine}), 7.42-7.46 (m, 1H, H_{pyridine}) 7.91-7.93 (m, 1H, H_{pyridine}) 8.04 (d, J=8.1, 1H, H_{pyridine}) 8.27 (d, J=9.9, 1H, H_{pyridazine}), 13.32 (bs, 1H, NH).

¹³C RMN (DMSO-d₆) δ (ppm): 118.8, 123.5, 129.3, 130.1, 136.8, 142.9, 148.5, 151.3, 160.1.

MS, m/z (I %): 173 (M⁺, 71%), 145 (M⁺-N₂, 10%), 117 (M⁺-CCONH₂, 100%).

### Example 14

### 3-chloro-6-(pyridin-2-yl)-pyridazine 8a

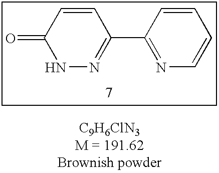 [0327] This compound is prepared according to Procedure C from 6-(pyridin-2-yl)-2H-pyridazin-3-one (7) (4.3 g, 24.8 mmol) and POCl₃ (30 mL). 3-chloro-6-(pyridine-2-yl)-pyridazine (8a) is obtained in the form of a solid brown substance (5.84 mg, quantitative).
This compound is prepared according to Procedure E from 2-bromopyridine (0.604 mL, 6.33 mmol), zinc chloride (1.05 g, 6.33 mmol), from butyllithium (6.33 mmol) and from 3-chloro-6-iodo-pyridazine (103) (942 mg, 3.95 mmol) and tetrakis(triphenylphosphine) palladium (0) (450 mg, 0.39 mmol). The expected product is obtained with a yield of 62%.

¹H RMN (CDCl₃) δ ppm: 7.37-7.42 (ddd, J=7.5, 4.5, 1.2, ¹H, H_{pyridine}), 7.63 (d, J=9.0, 1H, H_{pyridazine}), 7.89 (dt, J=7.8, 1.8, 1H, H_{pyridine}) 8.55 (d, J=9.0, 1H, H_{pyridazine}) 8.64 (d, J=8.7, 1H, H_{pyridine}), 8.71 (d, J=4.5, 1H, H_{pyridine})

¹³C RMN (CDCl₃) δ ppm: 121.5, 124.9, 126.9, 128.6, 137.2, 149.4, 152.3, 156.8, 157.8.

MS, m/z (I %): 191 (M⁺, 28%), 163 (M⁺-N₂, 5%), 128 (M⁺-(N₂+Cl), 100%).

### Example 15

### 3-bromo-6-(pyridin-2-yl)-pyridazine 8b

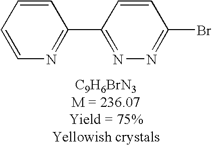

1 g (5.78 mmol) of 6-(pyridin-2-yl)-2H-pyridazin-3-one 7 and an excess (5 g) of phosphorus oxybromide are heated to reflux during 12 hours. The reaction mixture is poured into 100 mL of ice-water and neutralised with a dropwise aqueous solution saturated with NaHCO₃.

After extracting the dichloromethane (3×30 mL), the organic phase is dried over MgSO₄ and concentrated under reduced pressure, thus providing pyridazine bromide 8b with a yield of 75%. ¹H RMN (CDCl₃) δ ppm: 7.39-7.43 (ddd, 1H, J=0.9, 6.0, 7.5, H_{pyridine}); 7.76 (d, 1H, J=8.7, H_{pyridazine}); 7.88 (dt, 1H, J=1.8, 5.2, H_{pyridine}); 8.44 (d, 1H, J=8.7, H_{pyridazine}); 8.63 (d, 1H, J=8.1, H_{pyridine}); 8.70 (m, 1H, H_{pyridine}).

¹³C RMN (CDCl₃) δ ppm: 121.55, 125.08, 126.61, 132.01, 137.27, 148.35, 149.47, 152.48, 160.00.

SM, m/z (I %): 237 (M⁺+H, 23%), 235 (M⁺−H, 22%), 128 (M⁺-(Br+N₂), 100%).

### Example 16

### 6-(pyridin-2-yl)-2H-pyridazin-3-thione 10

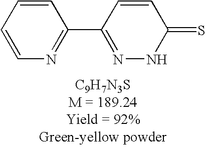

500 mg (2.9 mmol) of pyridazinone 7 and 772 mg (3.5 mmol) of phosphorus pentasulfide are dissolved in 20 mL of anhydrous pyridine. The reaction mixture is refluxed for 18 hours, and then cooled to room temperature. It is then poured in 200 mL of water and the resulting precipitate is filtered and washed with ice-water. Pyridazinethione 10 is obtained with a yield of 92%.

¹H RMN (CDCl₃) δ ppm: 7.37-7.39 (m, 1H, H_{pyridine}); 7.81-7.84 (m, 2H, H_{pyridine}, H_{pyridazine}); 8.15 (d, 1H, J=7.5, H_{pyridine}); 8.23 (d, 1H, J=8.7, H_{pyridazine}); 8.68 (d, 1H, J=4.2, H_{pyridine}); 12.34 (brs, 1H, NH).

¹³C RMN (CDCl₃) δ ppm: 120.08, 120.53, 120.55, 124.85, 137.14, 137.16, 137.53, 141.53, 149.36.

SM, m/z (I %): 189 (M⁺, 100%), 160 (M⁺, 35%).

### Example 17

### 6,6′-dimethoxy-3,3′-bipyridazine 15

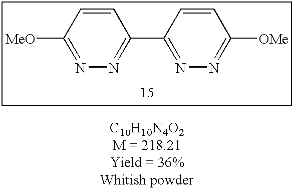

This compound is synthesised according to Procedure A, with tetrabutylammonium bromide (4.291 g, 13.31 mmol), zinc, powder activated (870 mg, 13.31 mmol), nickel (II) dibromobistriphenylphosphine (2.963 g, 3.99 mmol), and 3-chloro-6-methoxypyridazine (1.924 g, 13.31 mmol). At the end of the reaction, ammonia (25 N) is gently added and the medium is extracted with DCM. The organic phase is dried over Na₂SO₄, filtered and concentrated under reduced pressure. The compound is heated and recrystallised in ethanol, thus providing whitish crystals (494 mg) with a yield of 36%.

¹H RMN (CDCl₃) δ (ppm): 4.16 (s, 6H, OCH₃); 7.10 (d, J=9.3, 2H, H_{pyridazine}) 8.59 (d, J=9.3, 2H, H_{pyridazine}).

¹³C RMN (CDCl₃) δ (ppm): 54.9, 118.0, 127.2, 152.4, 165.3.

MS, m/z (I %): 218 (M⁺, 100%), 189 (M⁺-N₂, 22%), 175 (M⁺-(N₂+CH₃), 31%).

### Example 18

### 6,6′-bis(2,2-dimethylpyridin16-yl)-3,3′-bipyridazine

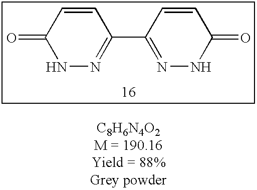

This compound is synthesised according to Procedure B with 6,6′-dimethoxy-3,3′-bipyridazine (15) (712 mg, 3.26 mmol) and a 33% HBr solution in acetic acid (4 mL). The obtained product is in the form of a greyish powder.

¹H RMN (TFA-d₁) δ (ppm): 7.35 (d, J=9.9, 2H, H_{pyridazine}), 8.38 (d, J=9.9, 2H, H_{pyridazine}).

¹³C RMN (TFA-d₁) δ (ppm): 130.7, 134.5, 146.1, 166.3.

MS, m/z (I %): 190 (M⁺, 100%), 175 (M⁺-N₂, 57%).

### Example 19

### 6,6′-dichloro-3,3′-bipyridazine 17

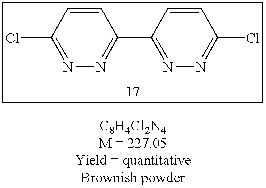

This compound is synthesised according to Procedure C, with 5 mL of POCl₃ and 1H,1′H-[3,3′]Bipyridazinyl-6,6′-dione (16) (541 mg), thus providing 6,6′-dichloro-[3,3′]bipyridazine (17) in the form of a brownish solid substance (584 mg, quantitative).

¹H RMN (CDCl₃) δ ppm: 7.73 (d, J=8.7, 2H, H_{pyridazine}); 8.77 (d, J=8.7, 2H, H_{pyridazine}).

¹³C RMN (CDCl₃) δ ppm: 126.9, 127.6, 129.2, 130.8.

MS, m/z (I %): 228 (M⁺+H, 27%), 227 (M⁺, 8%), 226 (M⁺−H, 41%), 163 (M⁺-(N₂+Cl), 100%).

### Example 20

### 6,6′-bis(pyridin-2-yl)-3,3′-bipyridazine 19

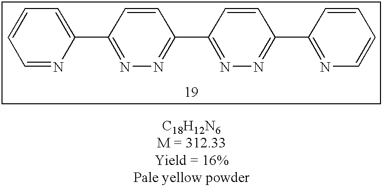 [0358] 6,6′-bis(pyridine-2-yl)-3,3′-bipyridazine (19) is prepared according to Procedure D. 6,6′-dichloro-[3,3′]bipyridazine (17), 2-tributylstannylpyridine (18), tetrakis(triphenylphosphine) palladium (0) and freshly distilled and degassed DMF are heated at 80° C. for 24 hours. The residue is heated and recrystallised in AcOEt, thus giving the expected result with a yield of 15%.
This compound is obtained according to Procedure A from 3-chloro-6(pyridin-2-yl)-pyridazine (8a) with a yield of 12%.
6,6′-bis(pyridine-2-yl)-3,3′-bipyridazine (19) is prepared according to Procedure E. 6,6′-dichloro-[3,3′]bipyridazine (17), 2-bromopyridine, zinc chloride, tetrakis(triphenylphosphine) palladium (0) and the freshly distilled and degassed DMF are heated at 80° C. for 48 hours. The residue is heated and recrystallised in AcOEt, thus giving the expected result with a yield of 16%.

¹H RMN (CDCl₃) δ (ppm): 7.45 (m, 2H, H_{pyridine}) 7.94 (dt, J=7.2, 1.8, 2H, H_{pyridine}) 8.79 (m, 6H, 2H_{pyridazine}, 4H_{pyridine}) 8.80 (d, J=9.0, 2H, H_{pyridazine}), 9.01 (d, J=9.0, 2H, H_{pyridazine}).

¹³C RMN (CDCl₃) δ (ppm): 121.8, 125.0, 125.4, 125.5, 137.3, 149.6, 153.1, 156.0, 159.2.

MS, m/z (I %): 312 (M⁺, 100%), 284 (M⁺-N₂), 255 (M⁺−2N₂, 55%), 91 (PyCH⁺, 89%).

UV-visible/Fluorescence (CH₂Cl₂): see FIG. 9.

### Example 21

### 6,6′-di-(1-ethoxyvinyl)-3,3′-bipyridazine 24

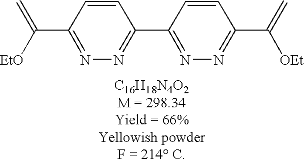

500 mg (2.21 mmol) of dichlorobipyridazine ( ), 1.591 g (4.42 mmol) of 1-ethoxyvinyl)tri(n-butyl)stannic, 77.4 mg (0.11 mmol) of dichlorobis(triphenylphosphine) palladium (II) and 50 mL of freshly distilled DMF are introduced in a 100 mL round-bottom flask fitted with a magnetic stirrer bar and a condenser. The reaction medium is stirred to reflux for 24 hours. After cooling to room temperature, the reaction medium is diluted with 80 mL of dichloromethane and poured in a KF saturated solution; after filtering, the residue is washed with an aqueous solution saturated with NaHCO₃, the organic phase is dried over MgSO₄ and concentrated under reduced pressure. The obtained residue is submitted to silica gel chromatography (elution of an ethyl acetate/petrol ether mixture) 6,6′-di-(1-ethoxyvinyl)-3,3′-bipyridazine (18) is isolated with a yield of 66%.

¹H RMN (CDCl₃) δ ppm: 51.47 (t, 6H, J=6.9 Hz, CH₃); 4.03 (q, 4H, J=6.9 Hz, OCH₂); 4.57 (d, 2H, J=2.4 Hz, H_{vinyl}) 5.85 (d, 2H, J=2.1 Hz, H_{vinyl}); 8.00 (d, 2H, J=9.0 Hz, H_{pyridazine}); 8.81 (d, 2H, J=8.7 Hz, H_{pyridazine}).

### Example 22

### 6-(pyridin-2-yl)-3-(tributylstannyl)-pyridazine 39

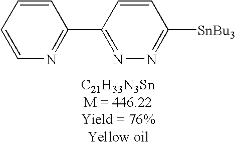

In a Schlenk tube fitted with a condenser, 1 g (5.24 mmol) of chloropyridazine 8a and 190 mg (0.79 mmol) of tetrakistriphenylphosphine palladium (0) are solubilised in 40 mL of freshly distilled DMF. The medium is degassed under cold conditions, and placed under vacuum. After a return to room temperature, 3.04 g (5.24 mmol) hexabutylditin are added. The solution is heated to reflux for 18 hours. The solvent is evaporated under reduced pressure and the residue is purified by neutral aluminium gel chromatography (elution: petrol ether/ethyl acetate=95/5), stannylpyridazine 39 is obtained with a yield of 76%.

¹H RMN (CDCl₃) δ ppm: 0.84-0.92 (m, 9H, CH₃); 1.19-1.37 (m, 12H, CH₂); 1.56-1.62 (m, 6H, CH₂); 7.33-7.37 (m, 1H, H_{pyridine}); 7.63 (d, 1H, J=8.4, H_{pyridazine}); 7.84 (dt, 1H, J=1.8, 7.6, H_{pyridine}); 8.35 (d, 1H, J=8.4, H_{pyridazine}); 8.67-8.72 (m, 2H, 2H_{pyridine}).

¹³C RMN (CDCl₃) δ ppm: 9.99, 13.52, 27.16, 28.87, 121.28, 121.36, 124.35, 134.12, 136.98, 149.15, 154.08, 156.38, 174.85.

### Example 23

### 3-(6-bromopyridin-2-yl)-6-(pyridin-2-yl)-pyridazine 43

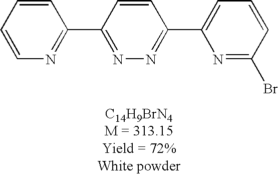

3-(6-bromopyridin-2-yl)-6-(pyridin-2-yl)-pyridazine 43 was prepared using the general Stille coupling procedure, starting from a mixture of 1.6 g (6.74 mmol) of 2,6-dibromobipyridine 42, 3 g (6.74 mmol) of 6-(pyridin-2-yl)-3-(tributylstannyl)-pyridazine 39, 546 mg (0.47 mmol) of tetrakis(triphenylphosphine) palladium (0) and 50 mL of freshly distilled toluene. The mixture is heated to reflux for 24 hours. The obtained residue is submitted to silica gel chromatography (elution: ethyl acetate/petrol ether=1/9). The bromide 43 product is isolated with a yield of 72%.

¹H RMN (CDCl₃) δ ppm: 7.40-7.44 (m, 1H, H_{pyridine}); 7.59 (dd, 1H, J=7.5, J=0.9, H_{pyridazine}); 7-76 (t, 1H, J=7.5, H_{pyridine}); 7.88-7.94 (m, 1H, H_{pyridine}); 8.64-8.76 (m, 5H, 4H_{pyridine}, 1H_{pyridazine}).

¹³C RMN (CDCl₃) δ ppm: 121.36, 121.90, 126.92, 124.97, 125.00, 125.48, 129.19, 133.12, 137.47, 139.50, 143.13, 149.36, 149.38.

SM, m/z (I %): 314 (M⁺+2, 92%), 312 (M⁺, 89%), 284 (M⁺-N₂, 35%), 205 (M⁺-(N₂+Br), 100%).

### Example 24

### 3-(6-methylpyridin-2-yl)-6-(pyridin-2-yl)pyridazine 44

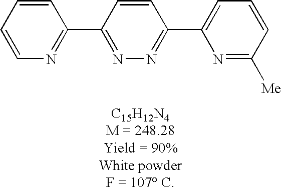

3-(6-methylpyridin-2-yl)-6-(pyridin-2-yl)-pyridazine 44 was prepared using the general Stille coupling procedure, starting from a mixture of 2 g (5.23 mmol) of 6-methyl-2-tributylstannylpyridine 22, 666 mg (3.49 mmol) of 3-chloro-6-(pyridin-2-yl)-pyridazine 8a, 208 mg of tetrakis(triphenylphosphine) palladium (0) and 50 mL of freshly distilled toluene. The reaction medium is stirred to reflux for 18 hours. The obtained residue is submitted to silica gel chromatography (elution: ethyl acetate/petrol ether=2/8). The coupling product 44 is isolated with a yield of 90%.

¹H RMN (CDCl₃) δ ppm: 2.62 (s, 3H, CH₃); 7.22 (d, 1H, J=7.8, H_{pyridazine}); 7.37 (ddd, 1H, J=0.9, 4.8, 7.5, H_{pyridine}); 7.76 (t, 1H, J=8.1, H_{pyridine}); 7-68 (dt, 1H, H_{pyridine}); 8-52 (d, 1H, J=7.8, H_{pyridazine}); 8.61-8.75 (m, 4H, 4H_{pyridine}).

¹³C RMN (CDCl₃) δ ppm: 24.49, 118.69, 121.66, 124.34, 124.69, 124.99, 125.19, 137.21, 137.35, 149.33, 152.60, 153.42, 157.89, 158.27, 158.33.

SM, m/z (I %): 248 (M⁺, 94%), 220 (M⁺-N₂, 100%) 205 (M⁺-(N₂+CH₃), 35%).

SMHR

Exact calculated mass [M+H]=249.1140;

Exact found mass [M+H]=249.1141.

### Example 25

### 3-(2-carboxypyridin-6-yl)-6-(pyridin-2-yl)-pyridazine 45

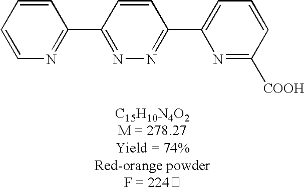

400 mg (2.42 mmol) of pyridazine 44, 177 mg (1.60 mmol) selenium dioxide, and 7 mL of o-dichlorobenzene are introduced in a round-bottom flask equipped with a condenser. The mixture is heated at 150° C. for 4 hours, and then cooled to room temperature. An excess of water is added to the formed precipitate, which is then filtered and washed with water. The obtained solid is dried to give an acid 45 with a yield of 74%.

¹H RMN (DMSO-d₆) δ ppm: 7.59 (ddd, 1H, J=1.2, 4.8, 7.5, H_{pyridine}); 8.07 (dt, 1H, J=2.1, 8.1, H_{pyridine}); 8.1-8.3 (m, 2H, H_{pyridine}); 8.64 (d, 1H, J=8.4, H_{pyridine}); 8.72 (d, 1H, J=9.0, H_{pyridazine}) 8.79 (m, 1H, H_{pyridine}) 8.82-8.85 (m, 2H, H_{pyridine}, H_{pyridazine}) 9.71 (brs, 1H, COOH).

¹³C RMN (DMSO-d₆) δ ppm: 121.1, 123.98, 125.09, 125.28, 125.48, 125.72, 137.65, 139.11, 148.32, 149.70, 152.53, 152.65, 157.12, 157.96, 165.64.

SM, m/z (I %): 279 (M⁺, 39%), 278 (M⁺−H, 100%), 250 (M⁺-N₂, 32%), 205 (M⁺-(N₂+COOH), 80%).

SMHR

Exact calculated mass [M]=278.0804;

Exact found mass [M]=278.0781.

### Example 26

### 3,6-bis(6-methylpyridin-2-yl)-pyridazine 47

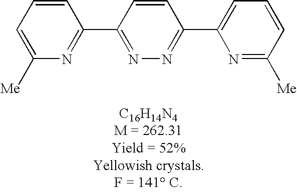

3,6-bis(6-methylpyridin-2-yl)-pyridazine 47 was prepared using the general Stille coupling procedure, starting from a reaction mixture of 1.55 g (4.06 mmol) of 6-methyl-2-tributylstannylpyridine 22, 300 mg (2.03 mmol) of 3,6-dichloropyridazine 46, 231 mg (0.20 mmol) of tetrakis(triphenylphosphine) palladium (0) and 50 mL of freshly distilled toluene. The reaction medium is stirred to reflux for 18 hours. and the obtained residue is purified by silica gel chromatography (elution: ethyl acetate/petrol ether=3/7). The disubstituted product 47 is isolated with a yield of 52%.

¹H RMN (CDCl₃) δ ppm: 2.65 (s, 6H, CH₃); 7.24 (d, J=6.3, 2H, H_{pyridine}); 7.77 (t, J=7.8, 2H, H_{pyridine}); 8.54 (d, J=8.7, 2H, H_{pyridine}) 8.68 (s, 2H, H_{pyridazine}).

¹³C RMN (CDCl₃) δ ppm: 24.35, 118.60, 124.18, 124.96, 137.14, 137.22, 152.78, 158.17, 158.21.

SM, m/z (I %): 262 (M⁺, 100%), 234 (M⁺-N₂, 85%), 142 (M⁺-(N₂+Py-CH₃), 48%).

SMHR

Exact calculated mass [M+H]=263.1297;

Exact found mass [M+H]=263.1317.

### Example 27

### 3,6-bis(2-carboxypyridin-6-yl)-pyridazine 48

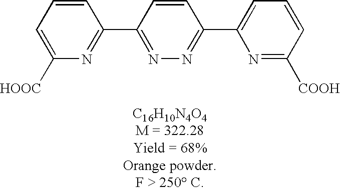

A diacid 48 is obtained according to a procedure that is similar to the preparation of an acid 45, starting from a 230 mg (0.88 mmol) mixture of pyridazine 47, 126 mg (1.14 mmol) of selenium dioxide, and 7 mL of o-dichlorobenzene. The mixture is heated at 150° C. for 12 hours, thus providing a diacid 48 with a yield of 68%.

¹H RMN (DMSO-d₆) δ ppm: 8.21-8.29 (m, 4H, H_{pyridine}); 8.82-8.85 (m, 2H, H_{pyridine}, H_{pyridazine}); 13.41 (1s, 2H, COOH).

¹³C RMN (DMSO-d₆) δ ppm: 124.13, 125.53, 125.83, 139.15, 148.38, 152.64, 157.38, 165.64.

SM, m/z (I %): 322 (M⁺, 100%), 294 (M⁺-N₂, 41%), 278 (M⁺-COOH, 47%).

SMHR

Exact calculated mass [M−H]=321.0624;

Exact found mass [M−H]=321.0623.

### Example 28

### 6-methyl-2-tributylstannylpyridine 22

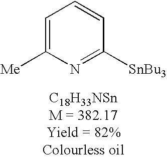

In a Schlenk tube at −10° C., 1.7 mL (2.6 mmol) of butyllithium (1.5 M in hexane) are added dropwise in 0.4 mL of a diisopropylamine (2.6 mmol) solution that is freshly distilled in anhydrous THF (50 mL). After 5 min, 0.70 mL (2.6 mmol) of tributyltin hydride. The stirring is maintained for 30 min at 0° C. A pale green solution is obtained a pale green solution of tributylstannyllithium, that will be cooled to −78° C., before dropwise addition of 294.5 μL (2.6 mmol) de 2-bromo-6-methylpyridine. The mixture is maintained for two hours at −78° C. After return to room temperature, the solvent is evaporated under vacuum. The residue is taken up in dichloromethane and washed with water. The organic phase is dried over MgSO₄, and dry evaporated. The product is purified by aluminium column chromatography (elution: petrol ether/ethyl acetate=0.5/9.5), by isolating stannylpyridazine 22 in the form of a yellow oil, with a yield of 82%.

¹H RMN (CDCl₃) δ ppm: 0.86-0.91 (m, 9H, CH₃); 1.07-1.12 (m, 12H, CH₂); 1.28-1.36 (m, 12H, CH₂); 1.44-1.59 (m, 12H, CH₂); 2.54 (s, 3H, CH₃); 6.95 (d, 1H, J=7.8, H_{pyridine}); 7-18 (d, 1H, J=7.5, H_{pyridine}) (t, 1H, J=7.5, H_{pyridine})

¹³C RMN (CDCl₃) δ ppm: 13.58, 13.67, 27.30, 27.81, 29.04, 120.63, 121.46, 129.32, 133.23, 158.53.

### Example 29

### Nickel (II) dibromobistriphenylphosphine

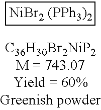

The monohydrated nickel bromide (4.37 g, 20 mmol) and the finely ground triphenylphosphine (10.48 g, 40 mmol) are dissolved separately in n-butanol (50 mL each). The solutions are refluxed until the reagents have completely dissolved. The solutions are then mixed in a heated environment. A greenish precipitate is formed and the reaction medium is stirred to reflux for 45 min and for 1 hour at room temperature. The solution is filtered and the precipitate is washed with 70 mL of n-butanol, 70 mL of ethanol and 70 mL of diethylic ether. After drying under vacuum, a greenish powder is obtained (8.85 g) with a yield of 60%.

### Example 30

### Tetrakis(triphenylphosphine) palladium (0)

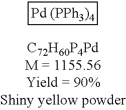

Palladium (II) chloride (0.9 g, 5.09 mmol) and finely ground triphenylphosphine (6.66 g, 25.42 mmol) are placed in a round-bottom flask fitted with an argon condenser after being dried under vacuum. The freshly distilled DMF (60 mL) is degassed cannulated into the reaction medium. The solution is stirred at 140° C. until clear. The solution is then cooled to 120° C. and the hydrazine (0.99 mL, 20.43 mmol) is added. A nitrogen release is immediately observed; this occurs simultaneously to the formation of a precipitate of a palladium (0) complex. After return to room temperature, the precipitate is filtered under vacuum and argon, washed with ethanol and diethylic ether, and dried under vacuum.

### Example 31

### 2-bromopicoline (104)

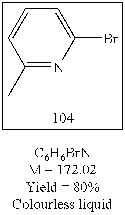

2-amino-picoline (37.35 g, 0.346 mol) is added to a mechanically-stirred round-bottom flask in several stages, in a hydrobromic acid solution (48% in water, 187 mL) at a temperature maintained between 20 and 30° C. After entire dissolution of the reagent, the reaction medium is cooled to 20° C. during the dropwise addition of dibromine (49 mL, 0.966 mol), for 30 min. The temperature of the solution is maintained at −20° C. for 90 minutes. In sodium nitrite solution (63.5 g, 6 mol) in water (100 mL) is added dropwise. The temperature of the solution is then brought to 15° C. in an hour and stirred for 45 minutes at that temperature. The medium is cooled to −20° C. and treated with a sodium solution (249 g, 400 mL H₂O) at a temperature maintained at below −10° C. during the adding. After return to room temperature, the solution is stirred for an hour and then extracted with AcOEt. The organic phase is dried over Na₂SO₄, filtered and concentrated under reduced pressure. The residue is distilled under vacuum and the 2-bromopicoline (104) is obtained in the form of a colourless oil with a yield of 80%.

bp 129-132° C. (2.6 mbar).

¹H RMN (CDCl₃) δ (ppm): 2.49 (s, 3H, H₇), 7.08 (d, J=7.6, 1H, H₅), 7.24 (d, J 7.6, 1H, H₃), 7.41 (t, 1H, H₄).

### Example 32

### 2-tributylstannylpyridine (18)

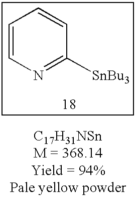

Butyllithium (2.5 M in hexane, 6.33 mmol) is added to a solution of 2-bromopyridine (1 g, 6.33 mmol) in THF (12 mL) freshly distilled and degassed at −78° C. The reddish solution is stirred for 30 minutes at −78° C. Tributyltin chloride (1.7 mL, 6.33 mmol) is then added and the solution is stirred for 1 hour −78° C. and for 1 hour at room temperature. The mixture is treated with a NH₄Cl saturated solution and extracted with diethylic ether. The organic phase is washed with a NaCl saturated solution, dried over MgSO₄ and concentrated under reduced pressure. The residue is submitted to aluminium column chromatography (hexane/AcOET: 20/1), thus providing a pure product with a yield of 94%.

¹H RMN (CDCl₃) δ (ppm): 8.73 (ddd, J=4.9, 1.9, 1.0, 1H, H₆), 7.48 (dt, J=7.4, 1.8, 1H, H₅), 7.39 (dt, J=7.4, 1.6, 1H, H₃), 7.10 (ddd, J=6.9, 4.9, 1.7, 1H, H₄), 1.70-1.05 (m, 18H, CH₂), 0.85 (t, 9H, J=7.3, CH₃).

### Example 33

### 3-methoxy-6-(pyridin-2-yl)-pyridazine (102)

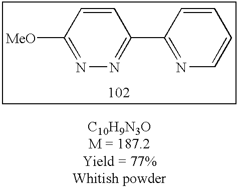

This compound is prepared according to Procedure D. 2-tributylstannylpyridine (18) (1.24 g, 3.36 mmol), 3-chloro-6-methoxypyridazine (0.37 g, 2.58 mmol), tetrakis(triphenylphosphine) palladium (0) (0.15 g, 0.13 mmol) and the freshly distilled and degassed toluene (19 mL) are placed under argon in a round bottom flask for 20 hours. After return to room temperature, the reaction medium is treated with a 15% HCl solution (2×30 mL), washed with diethylic ether, and a Na₂CO₃ saturated solution is added until an alkaline pH is achieved. The solution is extracted with DCM, the organic phase is dried over Na₂SO₄, filtered and concentrated under vacuum. The residue is purified by aluminium column chromatography (EP), thus providing the expected product with a yield of 77%.

¹H RMN (CDCl₃) δ (ppm): 4.2 (s, 3H, CH₃), 7.08 (d, J=9.7, 1H, H_{pyridazine}), 7-34 (m, 1H, H_{pyridine}), 7.83 (td, J=8.8, 1.7, 1H, H_{pyridine}), 8.47 (d, J=9.7, 1H, H_{pyridazine}), 8.57 (d, J=8.2, 1H, H_{pyridine}), 8.67 (d, J=5.5, 1H, H_{pyridine}).

### Example 34

### 2-bromo-4-methylpicoline (106)

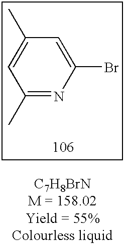

The procedure that is used is identical to 2-bromopicoline (104) applied to 2-amino-4-methylpicoline (10 g, 81.97 mmol)

¹H RMN (CDCl₃) δ (ppm): 2.28 (s, 3H, CH₃), 2.48 (s, 3H, CH₃—), 6.91 (s, 1H), 7.13 (s, 1H).

¹³C RMN (CDCl₃) δ (ppm): 20.5, 23.9, 123.3, 125.6, 141.3, 150.2, 159.4.

MS, m/z (I %): 185 (M⁺, 20%), 106 (M⁺-Br, 100%), 79 (Br⁺, 68%).

### Example 35

### 3-chloro-6-iodo-pyridazine (103)

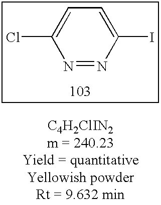

A mixture of 3,6-dichloropyridazine (46) (2 g, 13.42 mmol), of sodium iodide (2 g, 13.42 mmol), and hydroiodic acid (10 mL) in an argon atmosphere heated at 40° C. for 4 hours. After return to room temperature the reaction medium is poured onto ice, a concentrated sodium solution is added and the mixture is stirred for 10 minutes. The solution is then extracted with DCM. The organic phase is washed with water, dried over Na₂SO₄, filtered and concentrated under vacuum. La 3-chloro-6-iodo-pyridazine (103) is obtained in the form of a yellow powder (3.20 g, quantitative).

¹H RMN (CDCl₃) δ (ppm): 7.35 (d, J=9.0, 2H, H_{pyridazine}), 8.38 (d, J=9.0, 2H, H_{pyridazine}).

¹³C RMN (CDCl₃) δ (ppm): 122.9, 129.2, 139.2, 157.1.

MS, m/z (I %): 240 (M⁺, 18%), 127 (I⁺, 100%).

### Example 36

### 6,6′-bis(4,6-dimethylpyridin-2-yl)-3,3′-bipyridazine or 6,6′-dipicolin-4,4′-di-methyl-2-yl-[3,3′]bipyridazine (105)

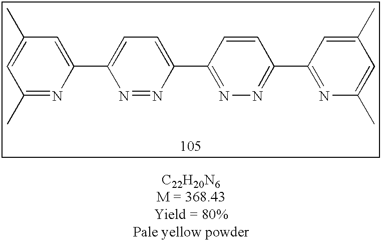

This compound is synthesised according to Procedure E from 2-bromo-4-methylpyridine (106) and from 6,6′-dichloro-[3,3′]bipyridazine (17). The expected product is obtained with a yield of 80% after hot recrystallisation in AcOEt.

¹H RMN (CDCl₃) δ (ppm): 2.46 (s, 6H, CH₃), 2.63 (s, 6H, CH₃), 7.13 (s, 2H, H_{pyridine}), 8.79 (d, J=8.9, 2H, H_{pyridazine}), 8.97 (d, J=8.9, 2H, H_{pyridazine})

MS, m/z (I %): 368.1 (M⁺, 100%), 340.1 (M⁺-N₂, 90%).

UV/Fluorescence (DCM): see FIG. 11.

### Example 37

### 5,5′-bis(pyridine-2-yl)-2,2′-bi(1H-pyrrole) (112)

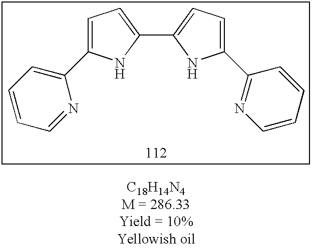

This compound is prepared according to Procedure F from 6,6′-bis(pyridine-2-yl)-3,3′-bipyridazine (19) and in a 0.5M H₂SO₄ solution used as solvent. The compound is obtained after purification on silica preparative plates (AcOEt).

RMN (CDCl₃) δ (ppm): 6.43 (d, J=3.5, 2H, H_{pyrrole}) 6.68 (d, J=3.5, 2H, H_{pyrrole}), 6.97-7.01 (m, 2H, H_{pyridine}), 7.43-7.61 (m, 4H, H_{pyridine}) 8.42 (d, J=7.8, 2H, H_{pyridine}).

### Example 38

### 6-(pyridin-2-yl)-3-[(5-pyridin-2-yl)-1H-pyrrol-2-yl]-pyridazine (107)

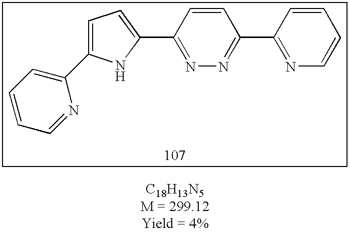

¹H RMN (CDCl₃) δ (ppm): 6.73 (m, 1H, H_{pyrrole}), 6.84 (m, 1H, H_{pyrrole}), 7.06 (m, 1H, H_{pyridine}) 7.29 (m, 1H, H_{pyridine}), 7.51-7.62 (m, 2H, H_{pyridine}), 7.72 (d, J=9.8, 1H, H_{pyridazine}), 8.43 (d, J=9.8, 1H, H_{pyridazine}), 8.46 (m, 2H, H_{pyridine}), 8.59-8.69 (m, 2H, H_{pyridine}) 10.89 (brs, 1H, NH).

### Example 39

### 5,5′-bis(3-methylpicoline-2-yl)-2,2′-bi(1H-pyrrole) (109)

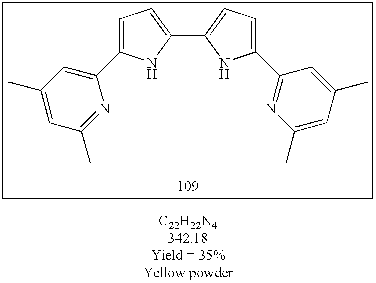

The compound is obtained from 6,6′-bis(4,6-dimethylpyridine-2-yl)-3,3′-bipyridazine (105) with the solvent system THF/Acetic buffer/CH₃CN: 5/4/1 (E=−1.05 V/ECS). The residue is submitted to chromatography on silica (AcOEt), the desired product is obtained with a yield of 35%.

¹H RMN (CDCl₃) δ (ppm): 2.32 (s, 6H, CH₃), 2.50 (s, 6H, CH₃), 6.45 (d, J=, 2H, H_{pyrrole}), 6.69 (d, 2H, H_{pyrrole}), 6.73 (s, 1H, H_{pyridine}), 7-18 (s, 2H, H_{pyridine}).

¹³C RMN (CDCl₃) δ (ppm): 20.9, 24.3, 29.7, 106.6, 108.4, 116.7, 121.1.

MS, m/z (I %): 342 (M⁺, 100%), 343 (M⁺+1, 100%).

### Example 40

### 6-(4,6-methylpyridin-2-yl)-3-{[5-(4,6-methylpyridin-2-yl)-1H-pyrrol-2-yl]-pyridazine} (110)

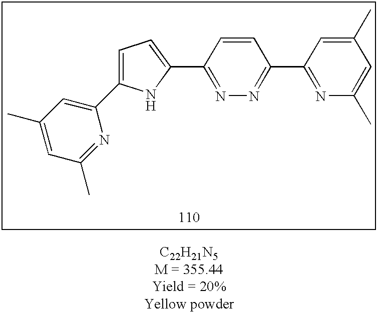

¹H RMN (THF-d₈) δ (ppm): 2.21 (s, 3H, CH₃), 2.30 (s, 3H, CH₃), 2.40 (s, 3H, CH₃), 2.43 (s, 3H, CH₃), 6.72 (m, 1H, H_{pyrrole}), 6.75 (s, 1H, H_{pyridine}), 6.85 (m, 1H, H_{pyrrole}), 6.98 (s, 1H, H_{pyridine}), 7.28 (s, 1H, H_{pyridine}), 7.79 (d, J=9, 1H, H_{pyridazine}), 8.23 (s, 1H, H_{pyridine}), 8.37 (d, J=9, 1H, H_{pyridazine}) 10.87 (bs, 1H, NH).

¹³C RMN (THF-d₈) δ (ppm): 19.5, 19.7, 23.0, 29.2, 108.4, 110.7, 117.9, 118.6, 121.1, 121.3, 123.5, 123.8, 128.2, 129.6, 130.2, 134.7, 138.2, 147.6, 148.8, 151.4, 152.8, 155.5, 157.1.

MS, m/z (I %): 356.2 (M+1, 23%), 355.2 (M⁺, 89%).

### Example 41

### 6-(4,6-methylpyridin-2-yl)-3-(5-(4,6-methylpyridin-2-yl)-1H-pyrrol-2-yl)-1,4,5,6-tetrahydropyridazine (111)

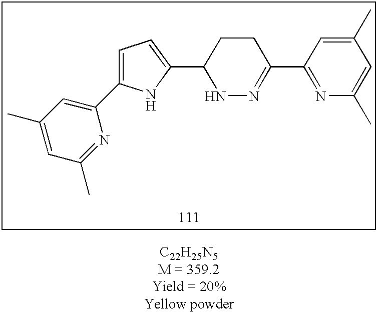

¹H RMN (THF-d₈) δ (ppm): 2.14 (s, 3H, CH₃), 2.17 (s, 3H, CH₃), 2.32 (s, 6H, 2CH₃), 2.14-2.49 (4H, Hterahydropyridazine), 4.08 (m, 1H, Hterahydropyridazine) 6.07 (m, 1H, H_{pyrrole}), 6.48 (m, 1H, H_{pyrrole}), 6.63 (s, 1H, H_{pyridine}), 6.78 (s, 1H, H_{pyridine}), 6.96 (s, 1H, H_{pyridine}), 7.12 (s, 1H, H_{pyridine}) 19.88 (bs, 1H, NH).

¹³C RMN (THF-d₈) δ (ppm):

MS, m/z (I %): 359.2 (M⁺, 45%), 355.2 (M⁺−2H₂, 78%).

### Example 42

### Therapeutic Activity of the Compounds According to the Invention

| | | KB cells | | | Parasitology IC_{50 or 80} | | | | | |
|---|---|---|---|---|---|---|---|---|---|---|
| | | Cytotoxicity | | | Leishmania | | Candida | | Aspergillus | |
| N^{o} of the | | % inhibition | IC₅₀ | IC₅₀ | mexicana | | albicans | | fumigatus | |
| compound | Structure | à [10⁻⁵] | μM | μg/mL | μM | μg/mL | μM | μg/mL | μM | μg/mL |

| | | | | | | | | | |
|---|---|---|---|---|---|---|---|---|---|
| 10 | 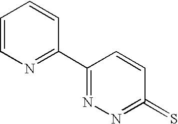 | 84 | 74 | | | | | | |
| 24 | 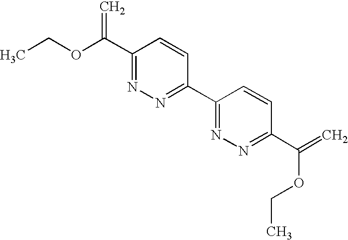 | 96 | 95 | 1.23 | 0.37 | | | | |
| 68 | 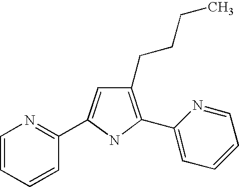 | 43 | | | | | | | |
| 45 | 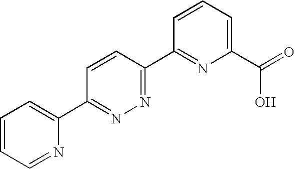 | 9 | 3 | | | 14 | 3.9 | >100 | |
| 44 | 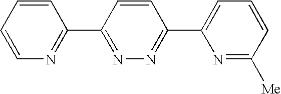 | 11 | 21 | | | <1 | <0.25 | 0.6 | 0.15 |
| 2 | 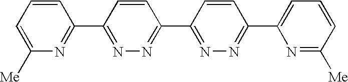 | 23 | 12 | | | 2.2 | 0.91 | 62 | 25.9 |
| 48 | 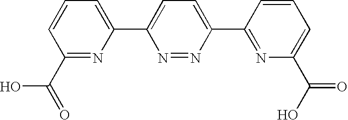 | 21 | | | | 3 | 0.96 | >100 | 32.2 |
| 19 | 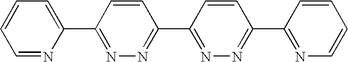 | 16 | | | | | | | |

### Example 43

### Therapeutic Activity of the Compounds According to the Invention

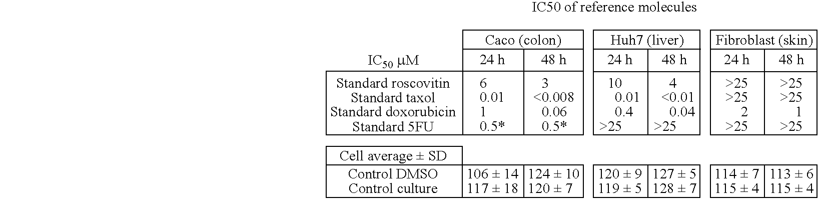

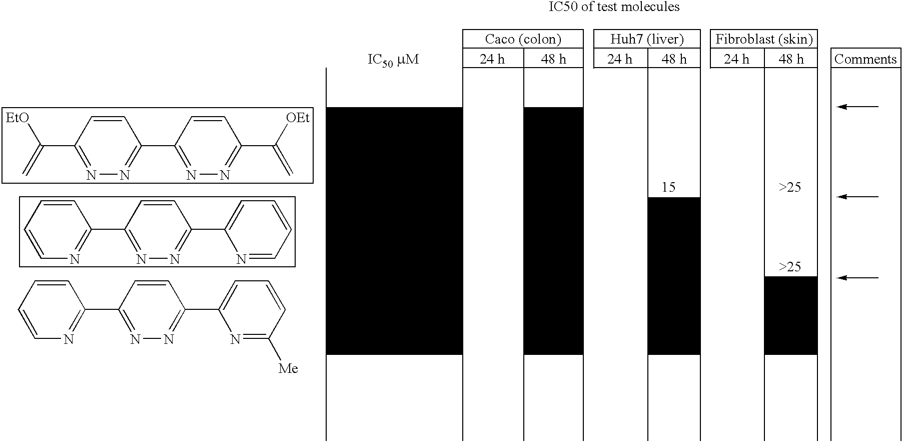

*= 20% inhibition

1. Compounds having the formula 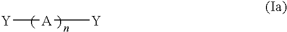
in which
if n=1
if A is a group of the formula 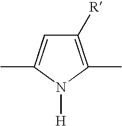
in which R′ is hydrogen, an alkyl, hydroxyalkyl, alkylamine, alkyloxy chain with 1-6 carbon atoms, a —COOH, —COOR1, —CONH₂, —CONHR1 group in which R1 is an alkyl chain with 1-6 carbon atoms,
the Y groups, which are identical or different, represent a group of the formula 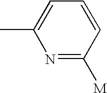
in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if A is a group of the formula 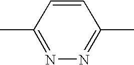
the Y groups, which are identical, represent a group of the formula 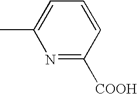
or the Y groups, which are different, represent a group of the formula 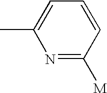
in which M is hydrogen, halogen, an alkyl, hydroxyalkyl, alkylamine or alkyloxy chain with 1-6 carbon atoms, a —COOH, —COOR1, —CONH₂, —CONHR1 group in which R1 is as defined above,
if n is an integer from 2 to 4, both inclusive,
the A groups, which are identical or different, represent a group of the formula 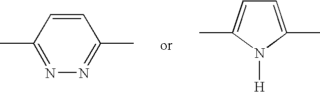
the Y groups, which are identical or different, represent halogen, hydroxy, mercapto, an alkyl, hydroxyalkyl, alkylamine or alkyloxy chain with 1-6 carbon atoms, optionally cyclic, a —COOH, —COOR1, —CONH₂, CONHR1 group in which R1 is as defined above, or a group selected from: 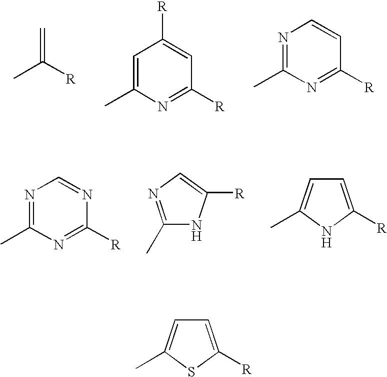
in which the R groups, which are identical or different, represent hydrogen, an alkyl, hydroxyalkyl, alkylamine or alkyloxy chain with 1-6 carbon atoms, a —COOH, —COOR1, —CONH₂, —CONHR1 group in which R1 is as defined above,
with the exception of the following compounds:
2,5-bis(pyridin-2-yl)pyrrole,
6,6′-bis(6-methylpyridin-2-yl)-3,3′-bipyridazine,
6,6′″-bis-(6-methylpyridin-2-yl)-[3,3′:6′,6″:3″,3′″]quaterpyridazine,
6,6′-dimethoxy-3,3′-bipyridazine,
6,6′-dichloro-3,3′-bipyridazine.

2-54. (canceled)
